(19) United States
(12) Patent Application Publication
(10) Pub. No.: US 2026/0054073 A1
(43) Pub. Date: Feb. 26, 2026

(54) EMBEDDED SEIZURE DETECTION DURING THERAPEUTIC BRAIN STIMULATION

(71) Applicant: Mayo Foundation for Medical Education and Research, Rochester, MN (US)

(72) Inventors: Victoria S. Marks, Shakiest, FL (US); Nicholas M. Gregg, Rochester, MN (US); Vaclav Kremen, Rochester, MN (US); Vladimir Sladky, Rochester, MN (US); Gregory A. Worrell, Rochester, MN (US)

(21) Appl. No.: 19/104,843

(22) PCT Filed: Aug. 21, 2023
(86) PCT No.: PCT/US2023/030710

§ 371 Date: Feb. 19, 2025

Related U.S. Application Data
(60) Provisional application No. 63/399,651, filed on Aug. 19, 2022.

(51) Int. Cl. A61N 1/36; G16H 50/30

(52) U.S. Cl. CPC A61N 1/36139; A61N 1/36064; A61N 1/36171; G16H 50/30

(57) ABSTRACT

This specification describes techniques for seizure detection in mammals. A first set of electrodes is used to stimulate a region of a brain of a mammal, and an electrical signal sensed by a second subset of electrodes is acquired during stimulation. The electrical signal is segmented into time-segmented portions according to a value of a time parameter. For each time-segmented portion of the electrical signal, a power is determined for the portion of the signal within a frequency band defined by a value of a frequency parameter. A classifier is used to classify the time-segmented portion of the signal as seizure positive or seizure negative based on the determined power within the frequency band. The values of the time and frequency parameters can result in the classifier achieving an effective level of performance defined by an area under a precision-recall curve (AU-PRC) for the classifier of at least 0.5.

### CROSS-REFERENCE TO RELATED APPLICATION

This application is a PCT Application which claims the benefit of U.S. Provisional Application No. 63/399,651 filed Aug. 19, 2022, the disclosures of which are incorporated herein.

### STATEMENT OF FEDERALLY SPONSORED RESEARCH

This invention was made with government support under NS073557, NS092882, and NS095495 awarded by the National Institutes of Health. The government has certain rights in the invention.

### BACKGROUND

### 1. Technical Field

This specification relates to brain stimulation and sensing, and more particularly to techniques for performing seizure detection based on intracranial electroencephalogram (iEEG) signals acquired in the presence of therapeutic brain stimulation.

### 2. Background Discussion

Epilepsy is a common neurological disorder. It has been estimated that the prevalence of epilepsy worldwide is as high as 621.5 per 100,000 people. Approximately one third of people with epilepsy have drug-resistant epilepsy (DRE). Many people with DRE are not candidates for epilepsy surgery or continue to have seizures despite surgery, and for these individuals, electrical brain stimulation is a viable treatment option.

There are currently two FDA approved implantable devices for epilepsy, but only approximately 15% of patients using these devices report extended seizure free periods. The data for tracking seizure outcomes in the pivotal trials is from patient diaries, which are notoriously inaccurate. Thus, evaluating the efficacy of brain stimulation is challenging.

Additional devices for electrical brain stimulation have been developed which are capable of both sensing intracranial (iEEG) signals and applying chronic or responsive brain stimulation. The sensing capabilities of these devices have primarily been directed to treatment of Parkinson disease, but the use of such devices for seizure detection has been relatively less explored.

### SUMMARY

This specification describes systems, methods, devices, and techniques for performing seizure detection based on iEEG signals. A detection unit can acquire iEEG signals from one or more electrodes implanted in a region of the patient's brain, e.g., the hippocampus or anterior nuclei of the thalamus (ANT). The iEEG signals can be segmented into fixed time intervals, each segment converted from the time domain to the frequency domain (e.g., using a fast-Fourier transform (FFT)), and the signal power within a specified frequency band can be computed for the segment. A classifier can then compare the computed signal power to a threshold power value, and the segment can be classified as seizure positive if the signal power meets or exceeds the threshold, or classified as seizure negative if the signal power does not meet or exceed the threshold. The detection unit can determine that the patient has experienced a seizure if one or more seizure positive segments are observed, and each detected seizure can be recorded in an electronic log that counts the number of seizures that have been detected over time. In some implementations, the log can further include timestamps that indicate the date and time at which each seizure event is detected. By electronically logging seizures, the need for manually maintained patient diaries can be reduced or eliminated, and a more reliable record of a patient's seizure history can be made available.

Moreover, performance of the classifier can be improved by sizing the length of the time-segmented iEEG signals and selecting a frequency band for power calculations that produces highly effective classification results with an appropriate threshold power value. The classifier can be further improved by applying different values for time and frequency parameters based on whether sensing is or is not performed in the presence of concurrent brain stimulation, and whether the brain stimulation is performed at a low- or high-frequency (e.g., 2 Hz thalamic stimulation or 145 Hz thalamic stimulation).

In one patient study, it was demonstrated that when stimulation was off, the classifier was most effective with a 35 Hz center frequency, 40 Hz bandwidth, and 30 second window for the time segments with hippocampal sensing (AU-PRC=0.692, 91.44% sensitivity, and 65 false positives per day). The study further demonstrated that, in the presence of 2 Hz thalamic stimulation, the classifier was most effective with a 5 Hz center frequency, 5 Hz bandwidth, and 20 second time segment window with hippocampal sensing (AU-PRC=0.571, 89% sensitivity, and 0.715 false positives per day). In the presence of 145 Hz thalamic stimulation, the classifier was most effective with a 17 Hz center frequency, 5 Hz bandwidth, and 20 second time segment window with hippocampal sensing (AU-PRC=0.626, 88.6% sensitivity, and 47 false positives per day).

In an aspect, a method is disclosed for detecting seizures in a mammal. The method can include stimulating, with a first set of electrodes in a brain of the mammal, a region of the brain; while stimulating the region of the brain, acquiring an electrical signal sensed by a second subset of electrodes in the brain; identifying values for a time parameter and a frequency parameter to be used in signal power calculations; segmenting the electrical signal into a plurality of time-segmented portions according to a value of the time parameter; for each time-segmented portion of the electrical signal: (i) determining a power of the time-segmented portion of the electrical signal within a frequency band defined by a value of the frequency parameter; and (ii) using a classifier to classify the time-segmented portion of the electrical signal as seizure positive or seizure negative based on the determined power within the frequency band.

This aspect, and others, can further include one or more of the following features.

Values for the time parameter and the frequency parameter can be selected to cause the classifier to achieve an effective level of performance. In this specification, effective classifier performance is use-case specific (e.g. having a high sensitivity can be the priority for an automated seizure diary (e.g. an electronic seizure log), but avoiding false positives could be prioritized for responsive stimulation). Accordingly, effective classifier performance can be defined in a number of ways including AU-PRC of a least 0.5 or at least 0.6; sensitivity of at least 70%, at least 80%, at least 90%; or false positive rates less than 1 per day, less than 10 per day, or less than 100 per day when monitoring occurs for substantially the entire day (e.g., at least 18 hours, 20 hours, 23 hours, or all 24 hours of the day).

The mammal can be a human.

The region of the brain stimulated with the first set of electrodes can be a region of the thalamus.

The region of the brain stimulated with the first set of electrodes can include an anterior nuclei of the thalamus.

The second subset of electrodes can produce the electrical signal responsive to brain activity in the hippocampus.

Identifying values for the time parameter and the frequency parameter can include selecting a set of values for the time parameter and the frequency parameter that are associated with a frequency with which the first set of electrodes stimulates the region of the brain. Different sets of values for the time parameter and the frequency parameter can be associated with different stimulation frequencies.

A first set of values for the time parameter and the frequency parameter can be associated with a low-frequency stimulation and a second set of values for the time parameter and the frequency parameter can be associated with a high-frequency stimulation.

The low-frequency stimulation can include 2 Hz stimulation, and the high-frequency stimulation can include 145 Hz stimulation.

The frequency with which the first set of electrodes stimulates the region of the brain can be adjusted from a first frequency to a second frequency. In response to such an adjustment, a second set of values for the time parameter and the frequency parameter can be used to segment the electrical signal and determine powers of time-segmented portions of the electrical signal in place of a first set of values for the time parameter and the frequency parameter. The second set of values can be associated with the second frequency and the first set of values can be associated with the first frequency.

Stimulating the region of the brain can include applying a chronic deep brain stimulation therapy to the region of the brain.

The classifier can be implemented on a device implanted in a body of the mammal.

The device can include a memory and can be configured to maintain a log of seizures detected over time. The device can log a seizure event in response to the classifier classifying one or more time-segmented portions of the electrical signal as seizure positive.

The log can include a count of seizure events detected over time. The log can further include timestamps associated in log entries with each seizure event detected over time.

The value for the time parameter can be a temporal segment length that defines a temporal length of the time-segmented portions of the electrical signal.

The value for the frequency parameter can include a center-frequency component and a bandwidth component.

The value for the frequency parameter can include an upper cutoff frequency component and a lower cutoff frequency component.

Using the classifier to classify the time-segmented portion of the electrical signal can include comparing the determined power of the time-segmented portion of the electrical signal within the frequency band to a threshold power value.

In response to detecting a seizure event based on identification of one or more seizure positive time-segmented portions of the electrical signal, one or more parameters by which the first set of electrodes stimulate the region of the brain can be adjusted.

In response to detecting a seizure event based on identification of one or more seizure positive time-segmented portions of the electrical signal, stimulation of a second region of the brain different from the region of the brain stimulated by the first set of electrodes can be initiated.

A focal region of the brain can be stimulated at a frequency of 2 Hz. The value of the time parameter can be 10 seconds, a center-frequency component of the value of the frequency parameter can be 5 Hz, and a bandwidth component of the frequency value can be 5 Hz.

A focal region of the brain can be stimulated at a frequency of 145 Hz. The value of the time parameter can be 20 seconds, a center-frequency component of the value of the frequency parameter can be 17 Hz, and a bandwidth component of the frequency value can be 5 Hz.

In an aspect, a method is disclosed for detecting seizures in a mammal. The method can include stimulating, with a first set of electrodes in a brain of the mammal, a region of the brain; while stimulating the region of the brain, acquiring an electrical signal sensed by a second subset of electrodes in the brain; identifying values for a time parameter and a frequency parameter to be used in signal power calculations; segmenting the electrical signal into a plurality of time-segmented portions according to a value of the time parameter; for each time-segmented portion of the electrical signal: (i) determining a power of the time-segmented portion of the electrical signal within a frequency band defined by a value of the frequency parameter; and (ii) using a classifier to classify the time-segmented portion of the electrical signal as seizure positive or seizure negative based on the determined power within the frequency band; and logging, in memory of a device implanted in the mammal, an indication of each seizure event detected in the mammal over a period of time, each seizure event corresponding to one or more time-segmented portions of the electrical signal that were classified as seizure positive.

In an aspect, a system is disclosed having one or more stimulating electrodes, one or more sensing electrodes, a stimulation unit, and a seizure detection unit. The one or more stimulating electrodes can be disposed in a brain of a mammal. The one or more sensing electrodes can be disposed in a brain of the mammal. The stimulation unit can be configured to generate and deliver stimulation signals to the stimulating electrodes to cause the stimulating electrodes to stimulate a region of a brain of the mammal. The seizure detection unit can include a memory, signal acquisition circuitry, a signal segmentation engine, a power analyzer, and a classifier. The memory can store one or more sets of values for a time parameter and a frequency parameter. The signal acquisition circuitry can be configured to acquire an electroencephalogram (EEG) signal sensed by the one or more sensing electrodes. The signal segmentation engine can be configured to segment the EEG signal into a series of time-segmented portions according to a value of a time parameter. The power analyzer can be configured, for each time-segmented portion of the EEG signal, to determine a power of the time-segmented portion within a frequency band defined by a value of a frequency parameter. The classifier can be configured, for each time-segmented portion of the EEG signal, to classify the time-segmented portion as seizure positive or seizure negative based on the determined power of the time-segmented power within the frequency band.

In an aspect, a system comprises circuitry configured to perform any of the methods disclosed herein. The circuitry can include software, hardware, digital electronics, analog electronics, or a combination of these.

In an aspect, one or more non-transitory computer-readable media are encoded with instructions that, when executed by one or more processors, cause performance of any of the methods disclosed herein.

Additional features and advantages will be apparent to one of ordinary skill in view of the specification, the figures, and claims.

### BRIEF DESCRIPTION OF DRAWINGS

FIG. 1 is a block diagram of an example brain stimulation and sensing system configured to perform seizure detection.

FIG. 2 is a flowchart of an example process for detecting seizures using power-in-band classification techniques in the presence of electrical brain stimulation.

FIGS. 3A-3RRRR depict plots indicating performance of a seizure classifier in an example study as measured by area under the precision-recall curve (AU-PRC) under different time and frequency parameters.

FIG. 4 depicts time and frequency plots of an iEEG signal, and illustrates example changes in the iEEG signal at the onset of a seizure.

FIG. 5 depicts time and frequency plots of an example iEEG signal, and the use of time and frequency parameters to selectively measure the power of a signal over a particular time and in a particular frequency band to be used for seizure detection.

FIG. 6 depict plots of the power-in-band measurements obtained over time in an example study with patients administered 2 Hz thalamic brain stimulation. Power-in-band was calculated using the best performing parameters generalized across patients in the left column, and commercial parameters were used for calculation of the right column. Physician-annotated detections are indicated by the top line of crosses, while detections by the algorithm are indicated by the second line of dots. Different amplitude thresholds were used for detection with each of the four subjects.

FIG. 7 depicts plots of the power-in-band measurements obtained over time in an example study with patients administered 145 Hz thalamic brain stimulation. Power-in-band was calculated using the best performing parameters generalized across patients in the left column, and commercial parameters were used for calculation of the right column. Physician-annotated detections are indicated by the top line of crosses, while detections by the algorithm are indicated by the second line of dots. Different amplitude thresholds were used for detection with each of the four subjects.

FIG. 8 depicts a table of data characteristics from each patient.

FIG. 9 depicts area under the precision-recall curve (AU-PRC) calculations used to evaluate thalamic seizure detection performance in an example study based on various permutations of time and frequency parameters. (A) AU-PRC Curve for Subject 1 with zoom of the peak (B) at 7 Hz. (C) AU-PRC Curve for Subject 3 left hemisphere with zoom of the peak (D) at 7 Hz. (E) AU-PRC Curve for Subject 3 right hemisphere with zoom of the peaks (F). (G) AU-PRC Curve for Subject 4 with zoom of the peaks (H). (I) Color of line indicates the time window parameter.

FIG. 10 depicts area under the average precision-recall curve (AU-PRC) calculations in a study of thalamic seizure detection. The most effective parameters across patients in this study were 5 Hz bandwidth, 7 Hz center frequency, and 5 or 10 s time window. (A) The AUPRC curve for all patients was found by averaging the AUPRC curves from FIG. 2. (B) Zooming in on the peak clearly shows that 5 and 10 s are the most effective time windows. (C) Time windows are represented by the colors of the legend.

FIG. 11 depicts plots of detector performance in an example study of thalamic seizure detection. For the generalized detection parameters, the final AU-PRC value was 0.90. The parameters optimized according to the techniques described herein performed better than state-of-the-art parameters for (A) Subject 1, (B) Subject 3 left hemisphere, (C) Subject 3 right hemisphere, and (D) Subject 4. Physician-annotated detections are indicated by the top line of crosses, while detections by the algorithm are indicated by the second line of dots. Different amplitude thresholds were used for detection with each of the four subjects.

FIG. 12 depicts plots of detector performance in an example study of hippocampal detection. For the generalized detection parameters, the final AU-PRC value was 0.692. The parameters optimized according to the techniques described herein (left column, panels A, C, E, G, and I) performed better than state-of-the-art parameters (right column, panels B, D, F, H, and J) for (A, B) Subject 1, (C, D) Subject 2, (E, F) Subject 3 left hemisphere, and (I, J) Subject 4, but not (G, H) Subject 3 right hemisphere. Physician-annotated detections are indicated by the top line of crosses, while detections by the algorithm are indicated by the second line of dots. Different amplitude thresholds were used for detection with each of the four subjects.

FIG. 13 depicts plots of the power-in-band measurements from the hippocampus obtained over time in an example study with patients administered no thalamic brain stimulation. Power-in-band was calculated using the best performing parameters for each patient in the left column, and commercial parameters were used for calculation of the right column. Physician-annotated detections are indicated by the top line of crosses, while detections by the algorithm are indicated by the second line of dots. Different amplitude thresholds were used for detection with each of the four subjects.

FIG. 14 depict s plots of the power-in-band measurements from the hippocampus obtained over time in an example study with patients administered 2 Hz thalamic brain stimulation. Power-in-band was calculated using the best performing parameters for each patient in the left column, and commercial parameters were used for calculation of the right column. Physician-annotated detections are indicated by the top line of crosses, while detections by the algorithm are indicated by the second line of dots. Different amplitude thresholds were used for detection with each of the four subjects.

FIG. 15 depicts plots of the power-in-band measurements from the hippocampus obtained over time in an example study with patients administered 145 Hz thalamic brain stimulation. Power-in-band was calculated using the best performing parameters for each patient in the left column, and commercial parameters were used for calculation of the right column. Physician-annotated detections are indicated by the top line of crosses, while detections by the algorithm are indicated by the second line of dots. Different amplitude thresholds were used for detection with each of the four subjects.

FIG. 16 depicts components of example computing devices and systems that can be implemented in the computer-based aspects of the techniques disclosed herein.

### DETAILED DESCRIPTION

### Overview

The present technology addresses one of the fundamental gaps in clinical epileptology, seizure catalogs. It is well established that patients and their families are poor reporters of seizures. Unfortunately, this makes the clinical management of patients challenging due to lack of accurate seizure counts. Clinicians must make adjustments in electrical brain stimulation therapy parameters and drugs without clear knowledge of seizure counts. This specification describes a novel seizure detection program that, when implemented, can substantially mitigate these problems through the following steps: 1) spatiotemporal and spectral characterization of human seizure and non-seizure states, 2) develop and evaluate the performance of a seizure detection program capable of being embedded on existing clinical devices, 3) evaluation of the performance of such a detector during concurrent thalamic electrical stimulation, and 4) development of a pipeline for prospective seizure detection embedded on devices implanted in human subjects.

There is a paucity of data to guide anterior nucleus of the thalamus (ANT) electrical brain stimulation (EBS). Next-generation EBS devices capable of both electrical stimulation and local field potential brain sensing are now available. The evolution of deep brain stimulation (DBS) devices from a primary cell battery device capable of continuous or duty cycle stimulation have evolved to include brain sensing, embedded processors, and rechargeable batteries. The FDA-approved MEDTRONIC PERCEPT™ DBS device provides constrained, bedside brain sensing of local field potentials and chronic-power within a fixed frequency band (PIB), recorded in 10-minutes averaged increments in ambulatory humans. Here, three patients with temporal lobe epilepsy were implanted with an investigational device providing full bandwidth (2-100 Hz), chronic intracranial local field potential (cLFP) recordings from bilateral ANT and hippocampi (Hc) in ambulatory humans.

The technology described in this specification allows for continuous cLFP recordings from behaving humans with epilepsy. The cLFP enable recording the patients' spontaneously occurring seizures. The recordings were first used to assess ANT PIB-based seizure detection, the feature available on the currently available clinical PERCEPT™. Using the cLFP recordings of spontaneous seizures and continuous interictal periods (data between seizures), including the range of human brain behavioral states (e.g. awake, slow-wave sleep, rapid-eye-movement sleep), the performance of seizure detectors is fully characterized based on cLFP recordings. For the PIB detector, the optimization parameters were PIB center frequency, bandwidth, and epoch duration. Performance was evaluated against epileptologist-confirmed Hc seizures and assessed by area under the Precision/Recall curve (PR-AUC).

### Systems, Methods, and Devices

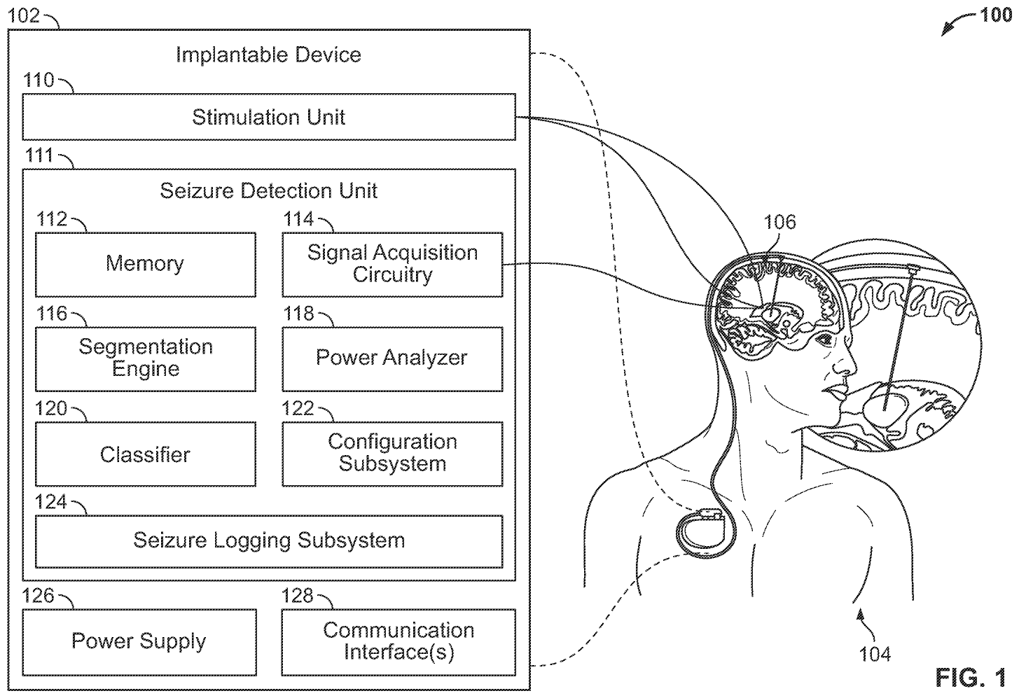

FIG. 1 is a block diagram of an example brain stimulation and sensing system 100 configured with seizure detection capabilities. The system 100 includes an implantable device 102, e.g., which may be implanted sub-dermally beneath a patient's collarbone. The patient 104 is a human in this example, although the techniques may be extended to other mammals as well. Implantable device 102 includes a stimulation unit 110, a seizure detection unit 111, a power supply 126 (e.g., a battery), and one or more communication interfaces 128. In some embodiments, not shown, the stimulation unit 110 and seizure detection unit 111 may be implemented in separate devices, and either or both of the stimulation unit 110 and seizure detection unit 111 may be operated without being implanted in the patient's body.

In general, stimulation unit 110 is configured to generate and deliver via wired leads electrical brain stimulation signals to stimulation electrodes in the patient's brain 106. The stimulation electrodes may be disposed in a region of the brain 106 to stimulate the anterior nuclei of the thalamus (ANT) or another brain region. The stimulation unit 110 can be an implantable pulse generator, and can deliver chronic brain stimulation, responsive stimulation, adaptive stimulation, or a combination of these.

Detection unit 111 includes a number of components for acquiring/sensing iEEG signals, analyzing iEEG signals to detect when seizures occur, and recording information in an electronic log about detected seizures. Signal acquisition circuitry 114 is electrically coupled via wired lead(s) to one or more sensing electrodes in the patient's brain 106. The sensing electrodes may be disposed in the hippocampus, the thalamus, the amygdala, the cingulate, or other focal regions of the brain where seizures may be generated. In some implementations, the sensing electrodes and stimulating electrodes are implanted in different brain regions, e.g., with stimulating electrodes in the thalamus and sensing electrodes in the hippocampus. In other implementations, the sensing electrodes and stimulating electrodes are implanted within a same region of the brain (e.g., thalamus). Although the sensing electrodes may be distinct from the stimulating electrodes in certain implementations, in other cases the same set of electrodes (or a subset of electrodes) may be used both for stimulation and sensing. The dual use of an electrode for stimulation and sensing can be achieved, for example, through interleaved sampling in which the same electrode is alternately used to deliver one or more stimulation pulses between sensing periods in which the electrode produces an iEEG signal. The signal acquisition circuitry 114 can continuously or otherwise chronically acquire iEEG signals to provide for continuous seizure monitoring. Signal acquisition circuitry 114 may include amplifiers, filters, and an analog-to-digital (A/D) converter, for example, to obtain digitized versions of iEEG signals that can be processed with digital processing components of the detection unit 111.

Memory 112 stores one or more configuration profiles defining values for time and frequency parameters that can be used together achieve an effective or optimized seizure detection capability. In some implementations, multiple configuration profiles are provided, each defining a different set of time and frequency parameter values for use in different conditions, e.g., depending on whether stimulation is performed, the frequency of stimulation, personalized patient characteristics, or other factors. The value of the time parameter defines a fixed length of time for iEEG signal segments, while the value of the frequency parameter defines a frequency band for power calculations. The value of the frequency parameter can be expressed in any suitable form, such as by a center frequency and bandwidth around the center frequency or by expressing the upper and lower cutoff frequencies for the band.

Segmentation engine 116 is operable to divide the iEEG signal into fixed-length time segments according to the value of the time parameter for the selected configuration profile, which is selected by the configuration subsystem 132 automatically or in response to user inputs. Power analyzer 118 is operable to calculate the signal power in each fixed-length time segment of the iEEG signal using a power-in-band technique. In some implementations, the signal segment is converted from the time domain to the frequency domain using a fast-Fourier transform, and the signal power is computed from the converted signal segment by computing the amount of power in the segment within a frequency band defined by the value of the frequency parameter for the selected configuration profile. Any suitable technique may be used to calculate the in-band power according to the selected parameters. For example, the studies referenced in this specification employed an averaged-power calculation, but other power calculations may also be employed such as summing the total power in the selected frequency band and time window.

Classifier 120 compares the computed power for the signal segment to a threshold power value. The segment is classified as seizure positive (and hence indicative of a detected seizure) if the computed power for the segment meets or exceeds the threshold power value. Otherwise, the segment is classified as seizure negative (and hence not indicative of a detected seizure) if the computed power for the segment does not meet or exceed the threshold power value. The threshold power value applied for a given system may vary from patient to patient due to the uniqueness of each patient's brain physiology, and may be calibrated to maximize the efficacy of the classifier. For example, after a system is initially implanted in a patient, power-in-band values from the patient's iEEG may be recorded for a period of time (e.g., one month) before the seizure detector and classifier are activated. The recorded PIB values could then be used to select a threshold value to be used by the classifier going forward to achieve an effective level of performance (e.g., 80% sensitivity, AU-PRC of at least 0.5 or 0.6, and/or <1 false positive per day).

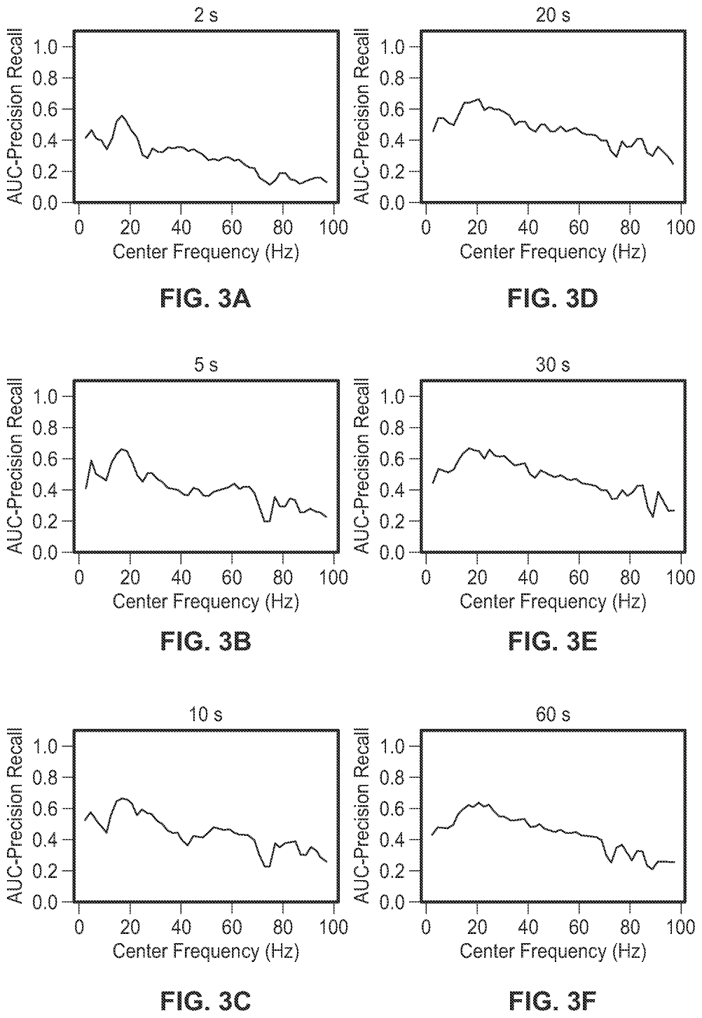

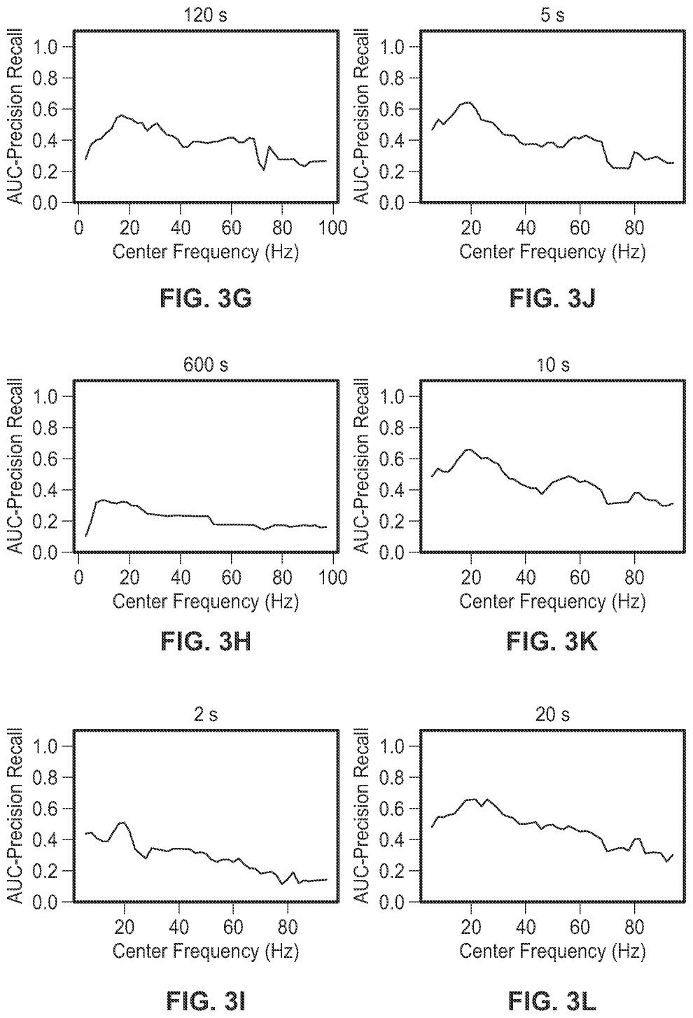

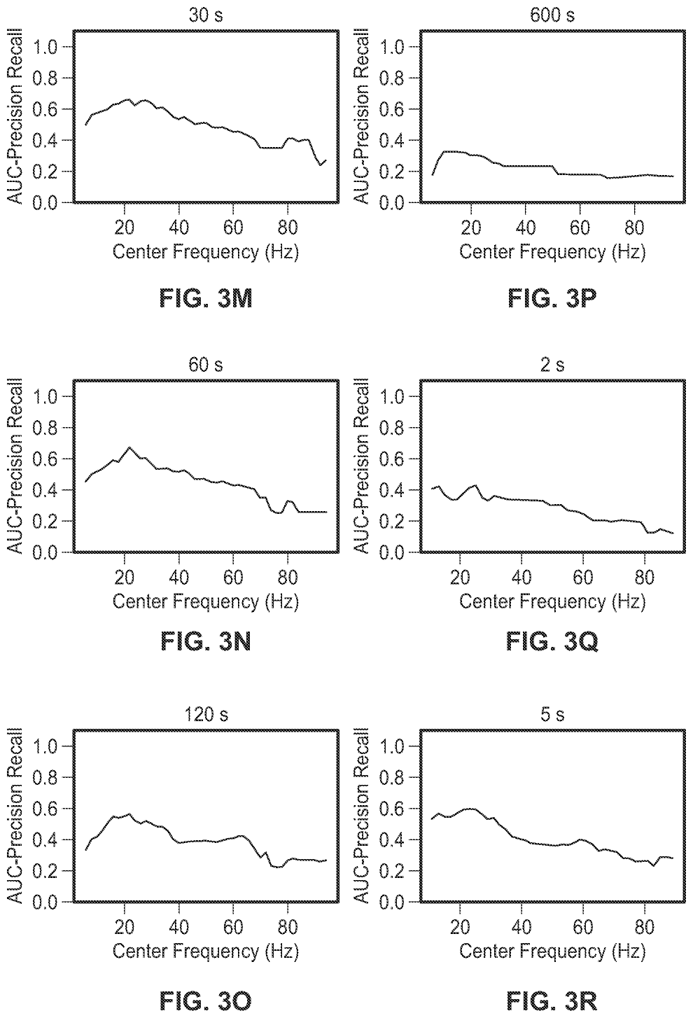

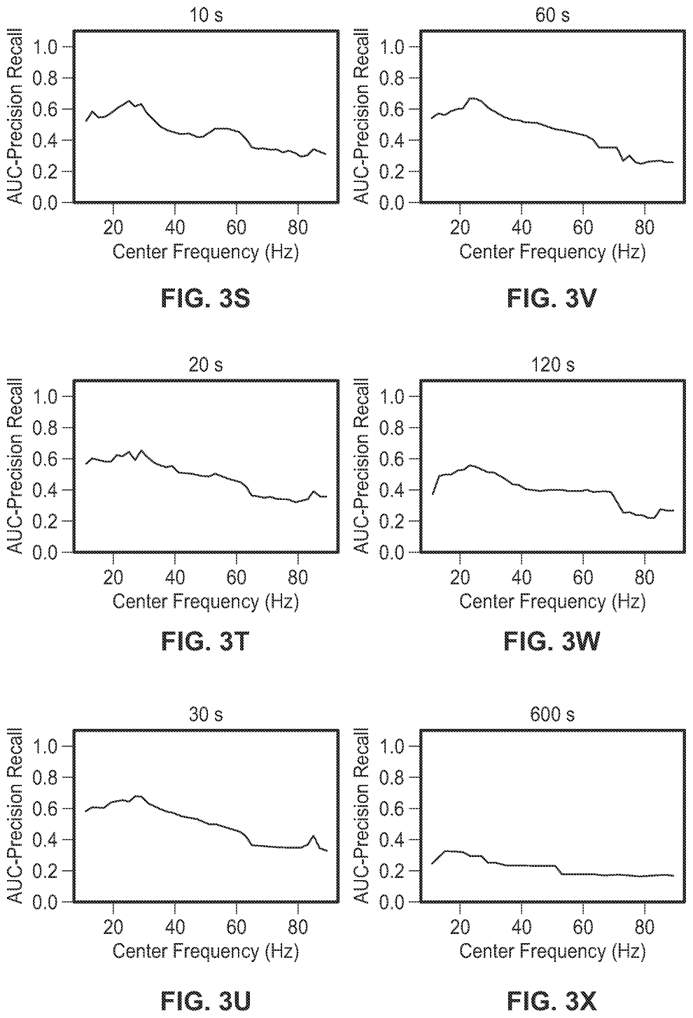

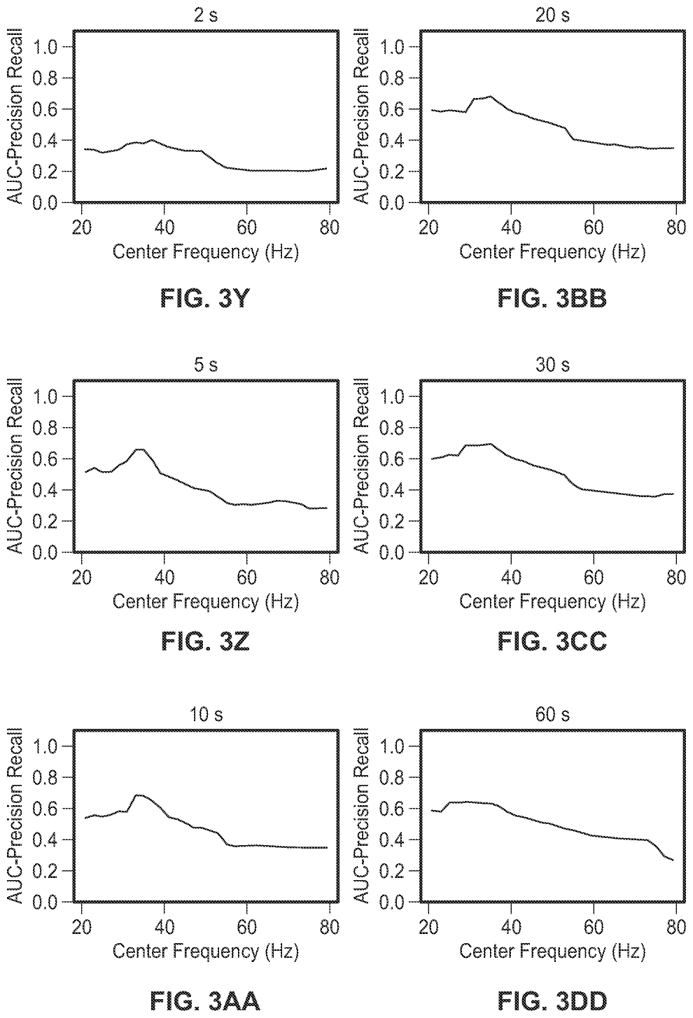

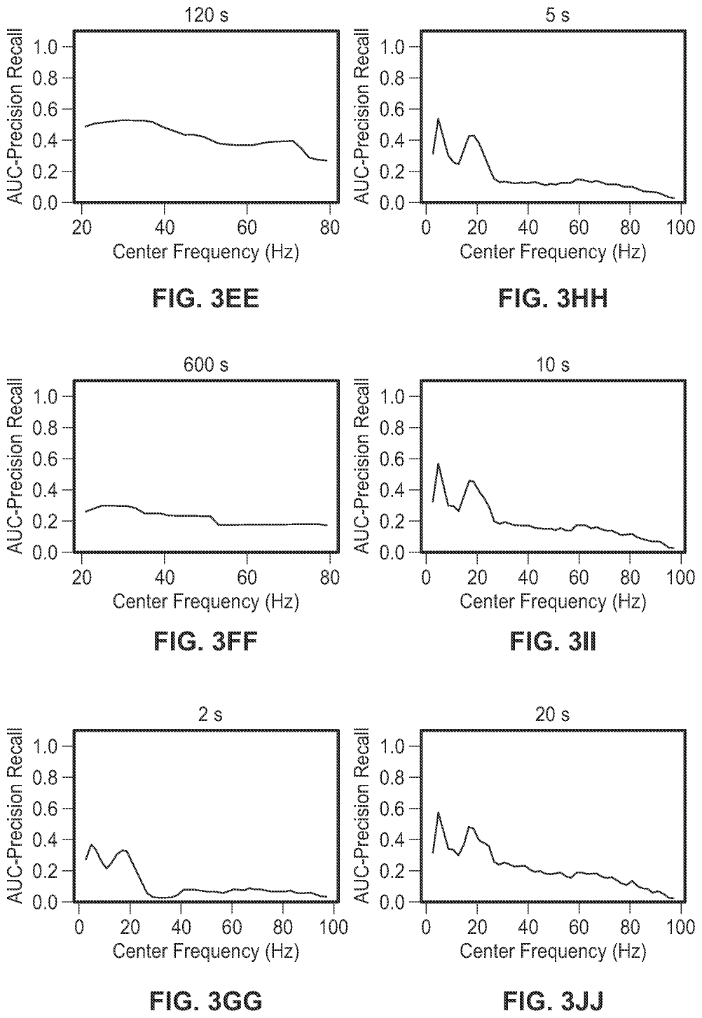

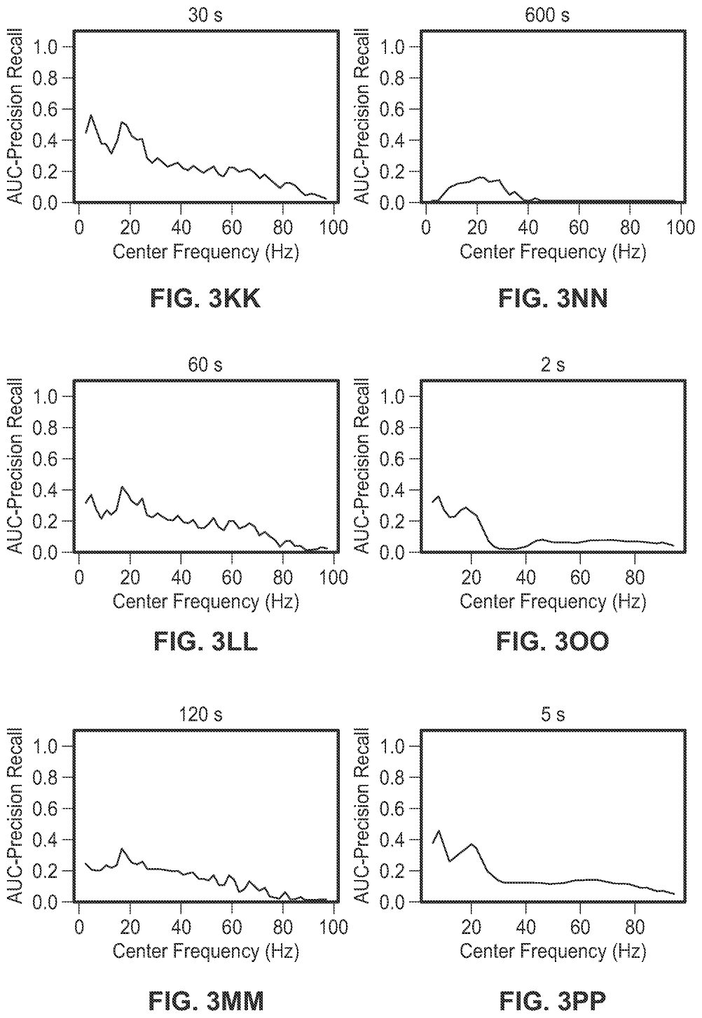

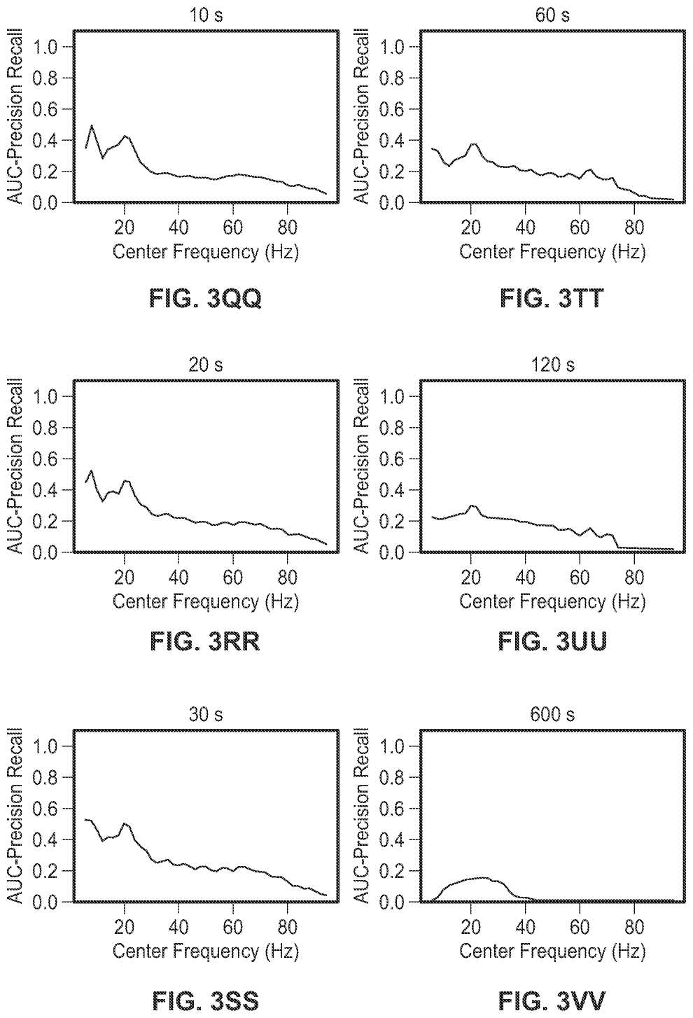

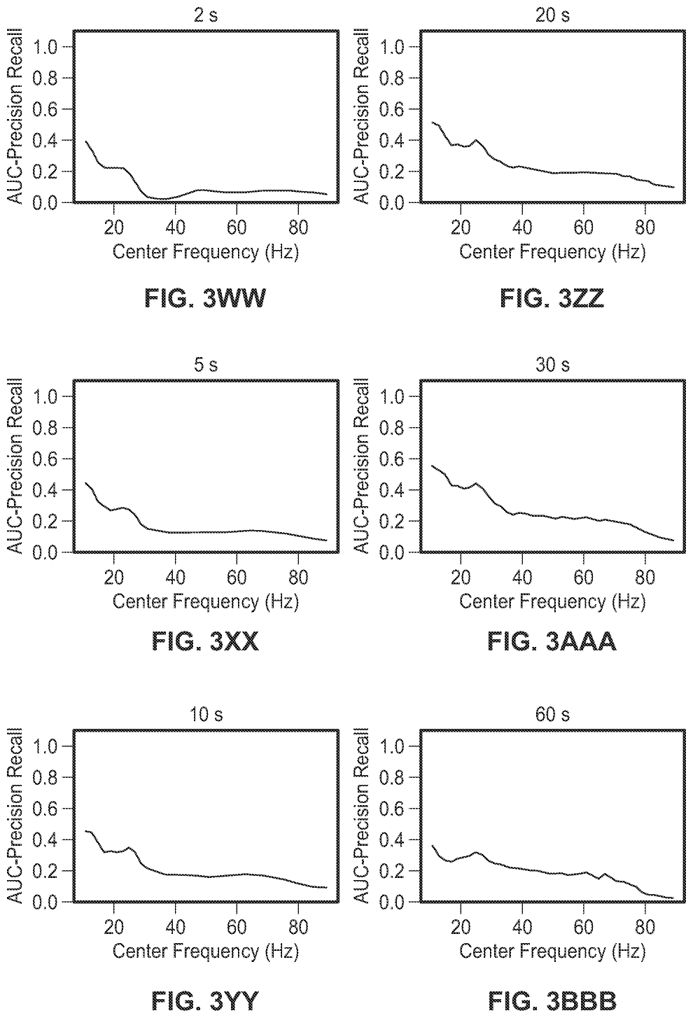

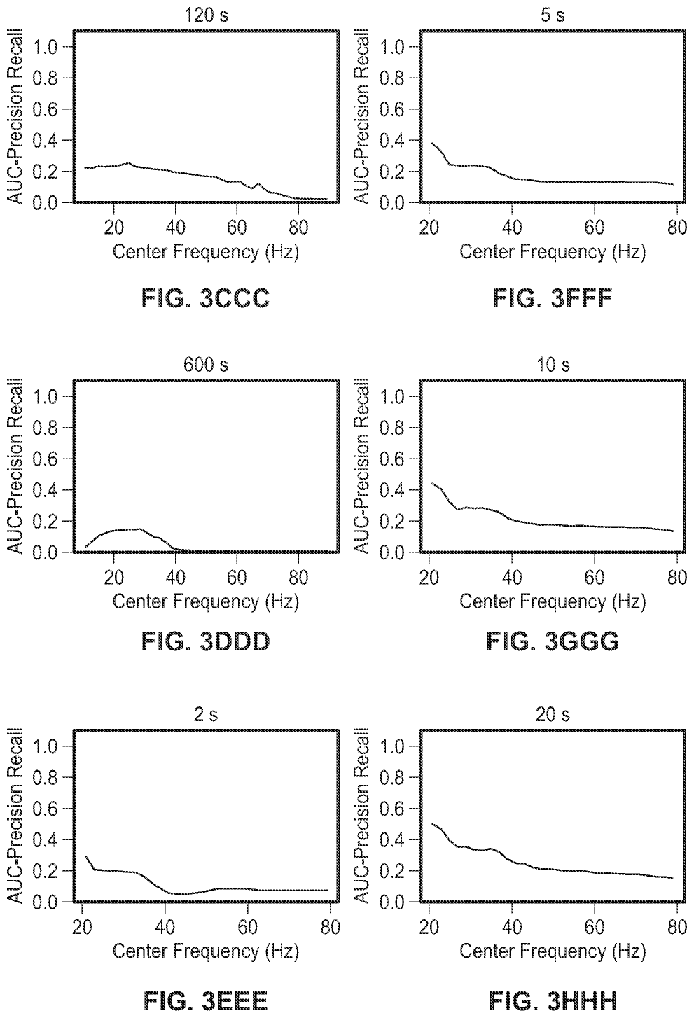

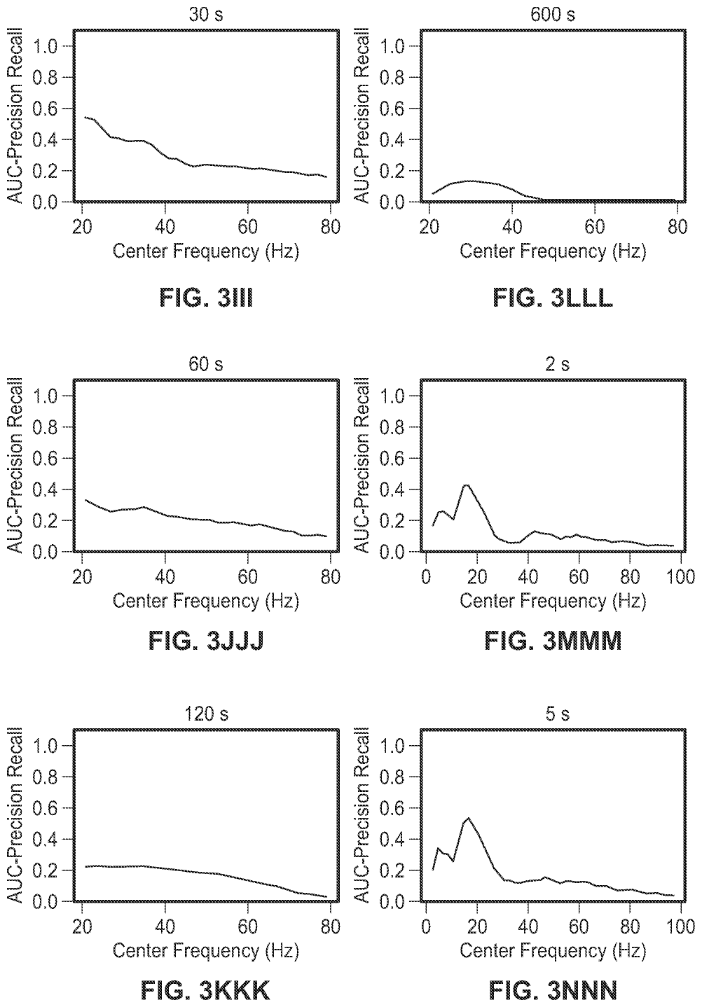

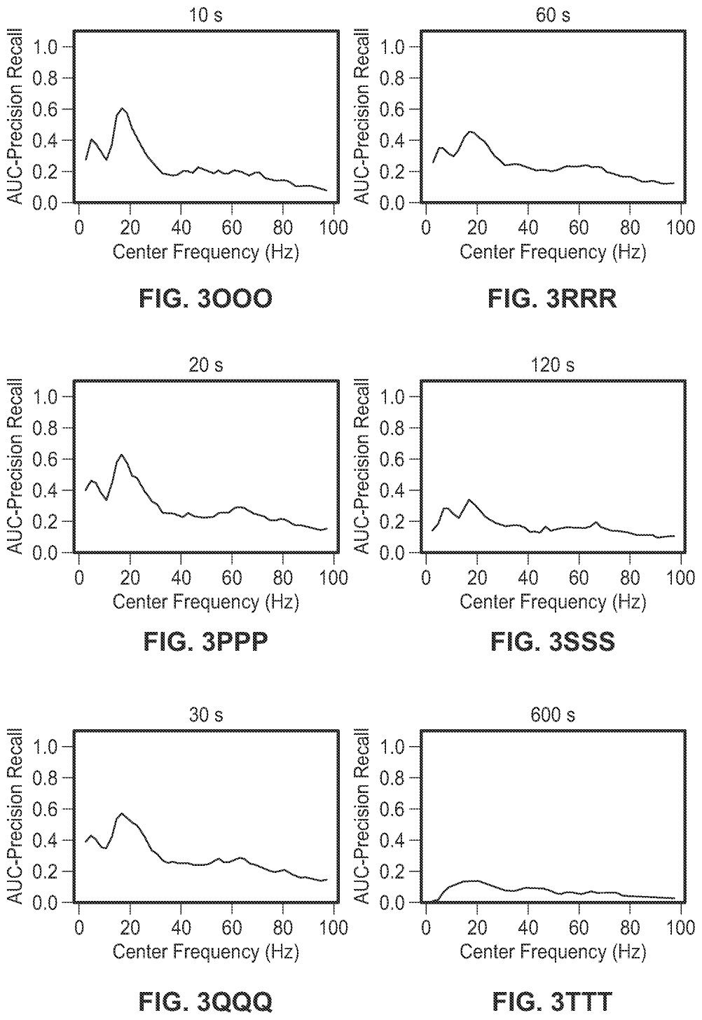

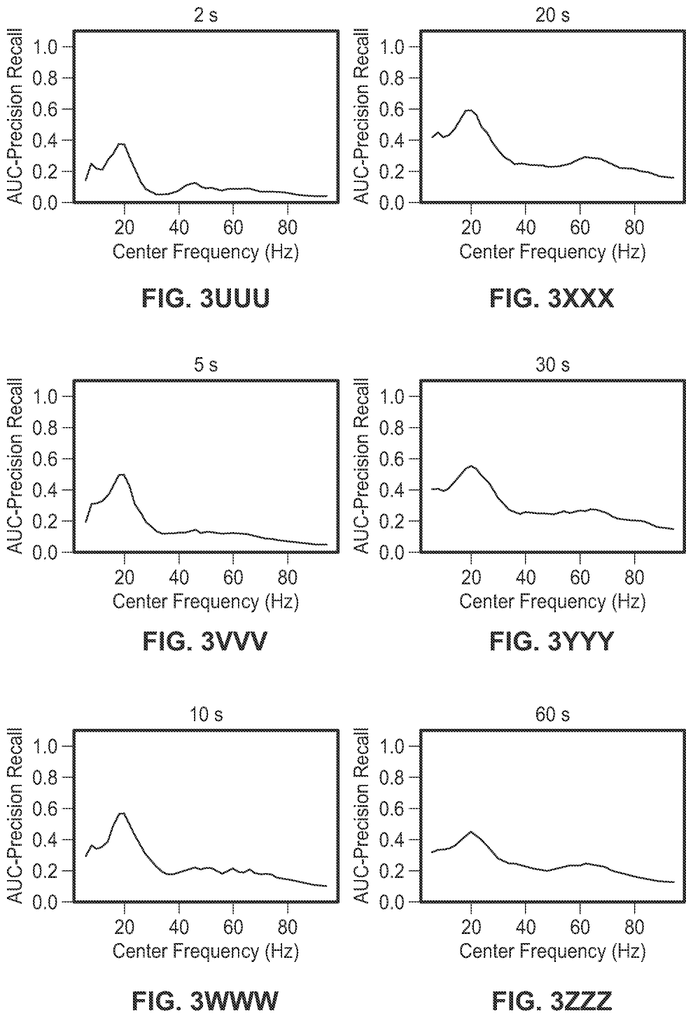

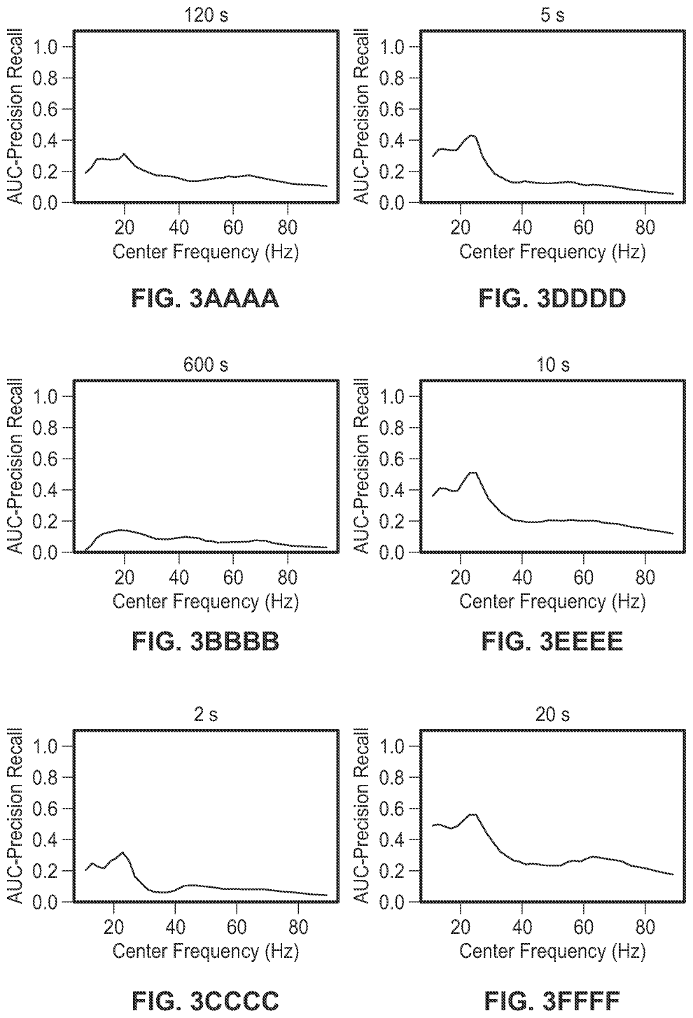

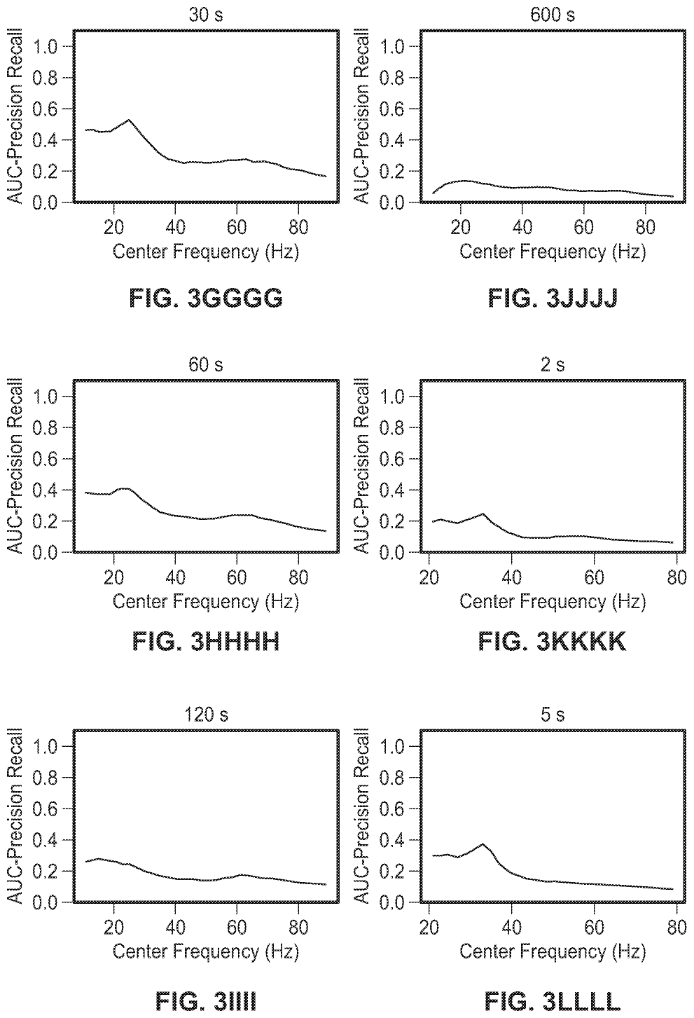

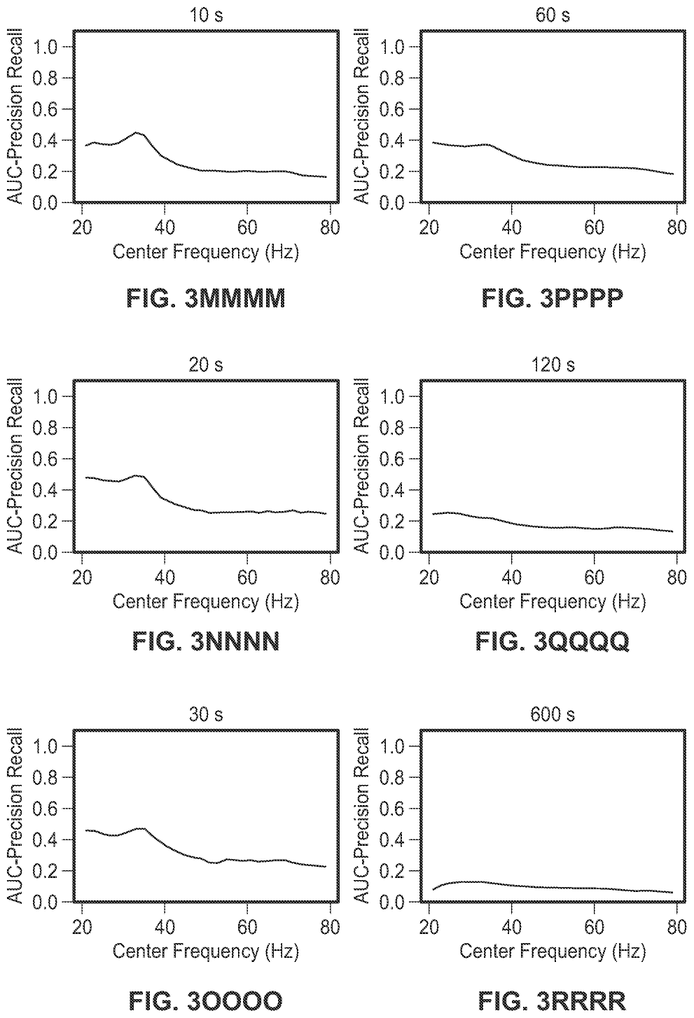

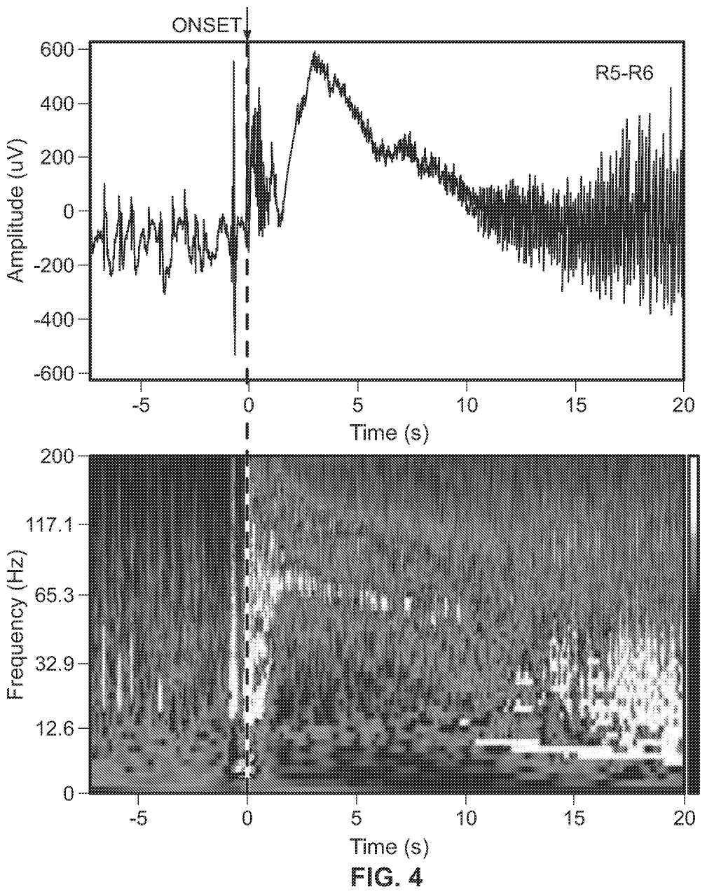

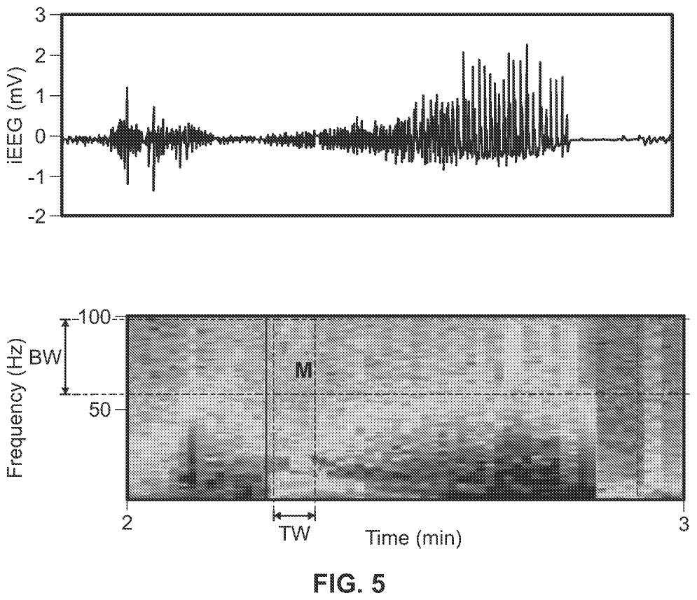

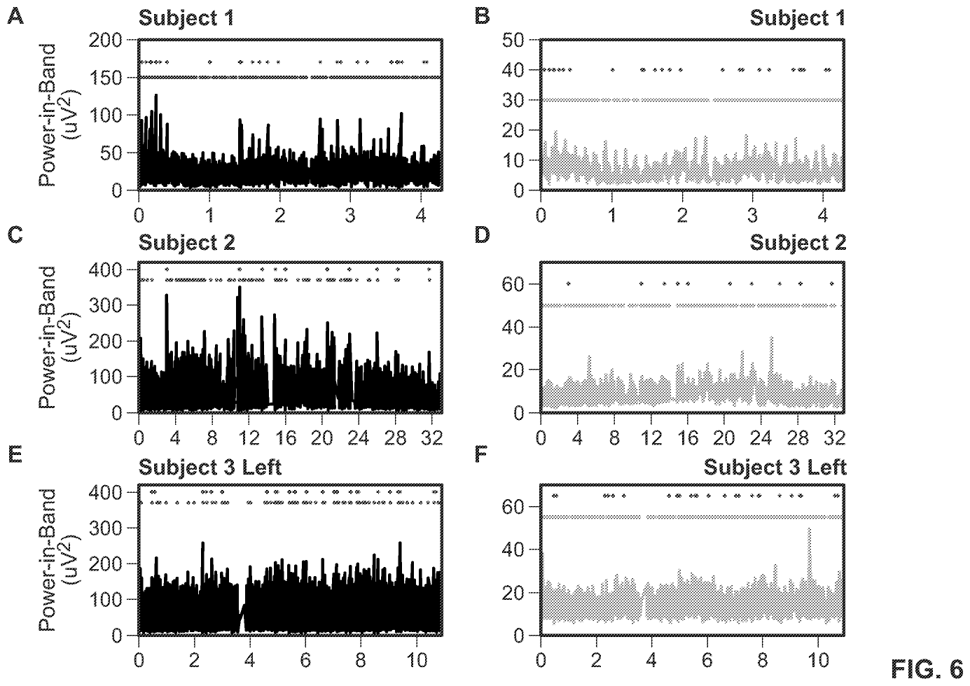

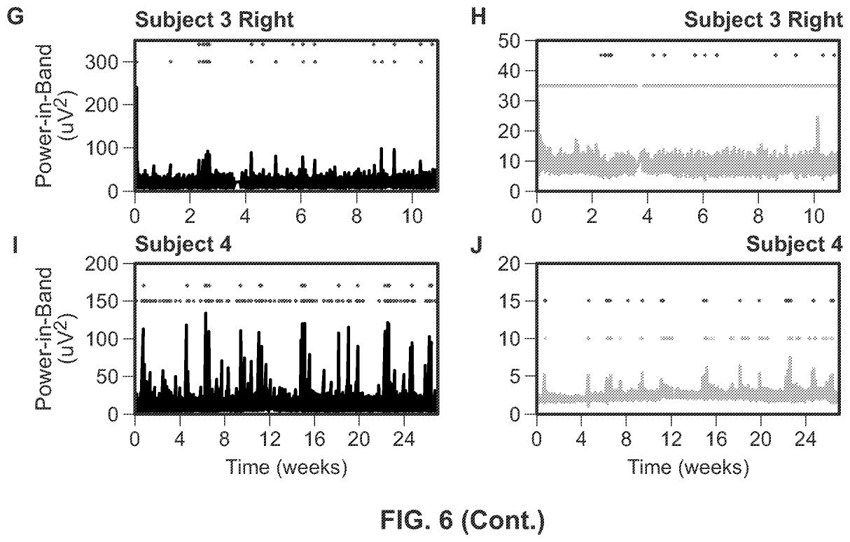

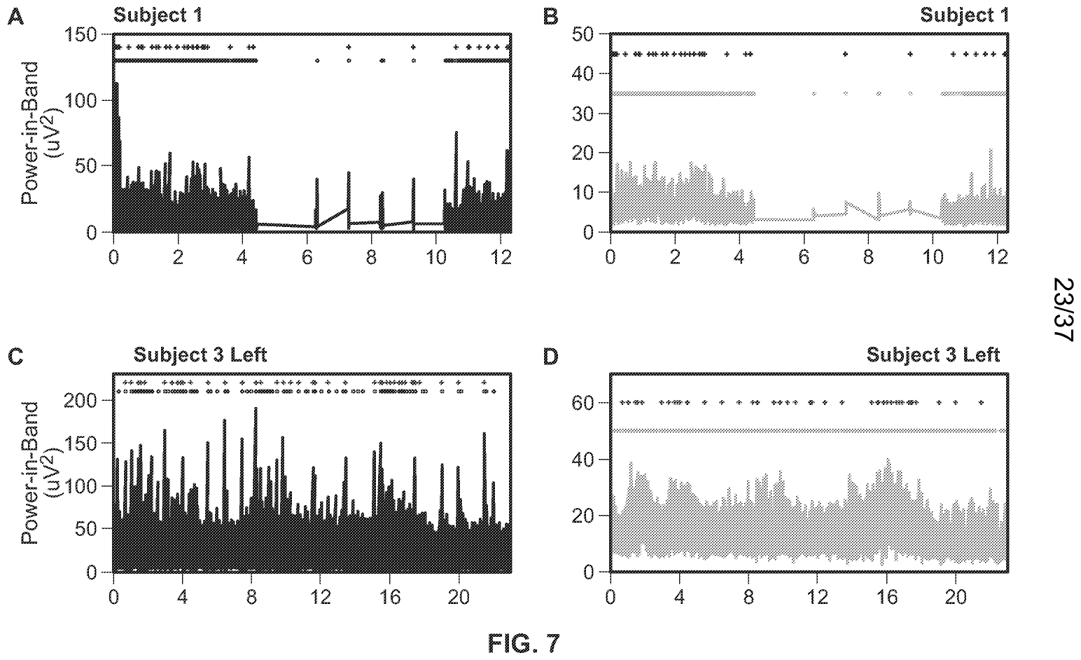

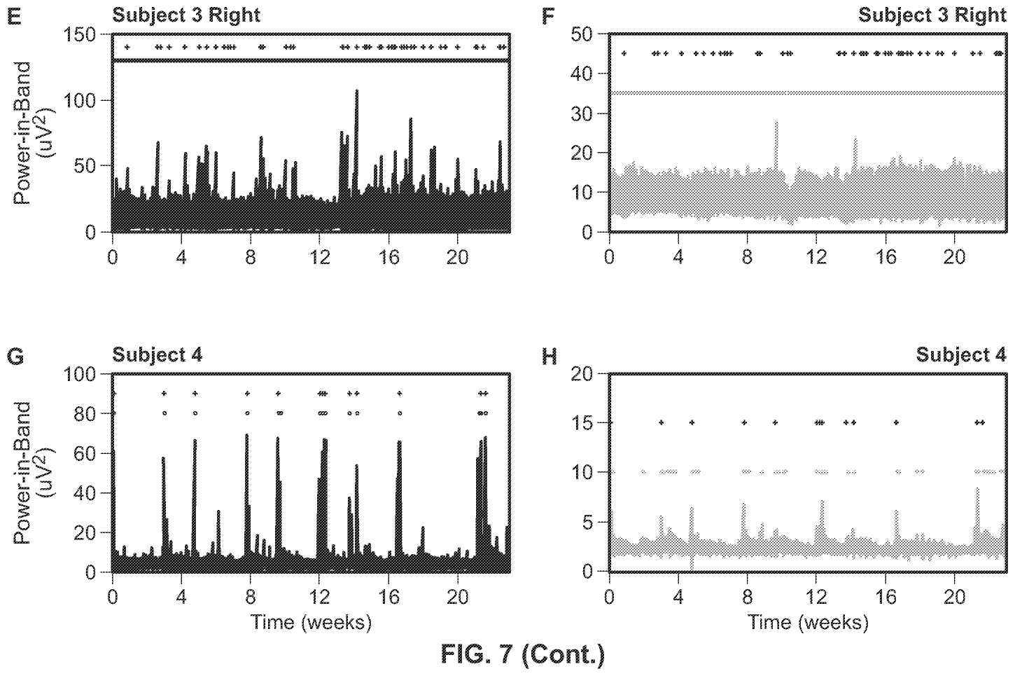

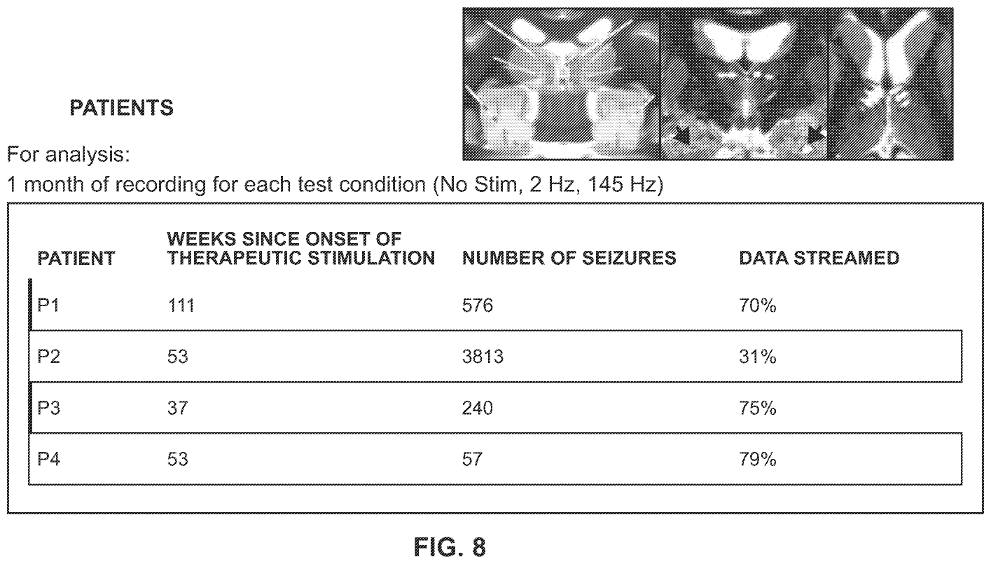

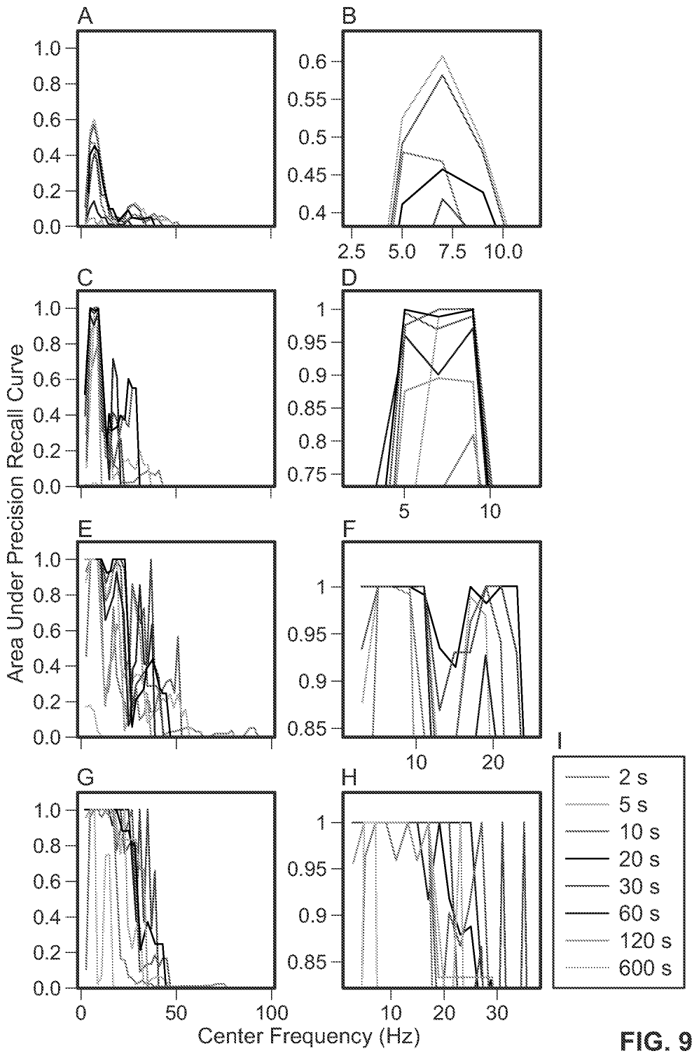

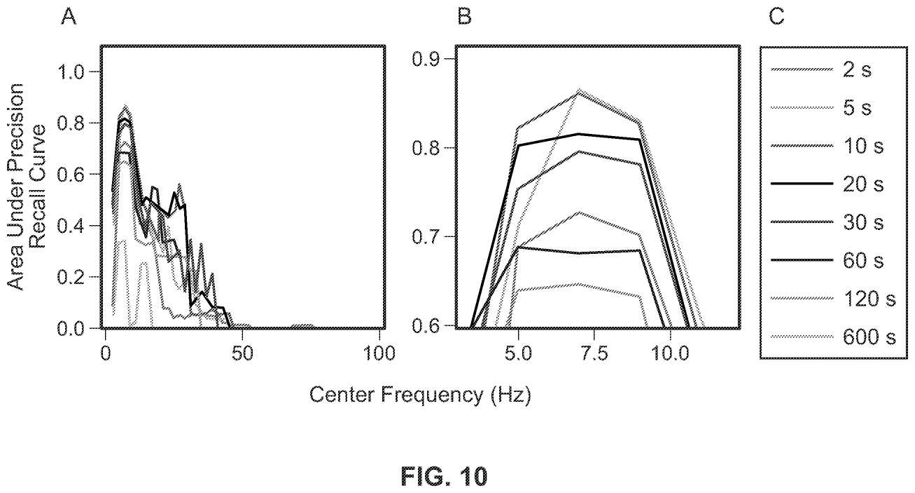

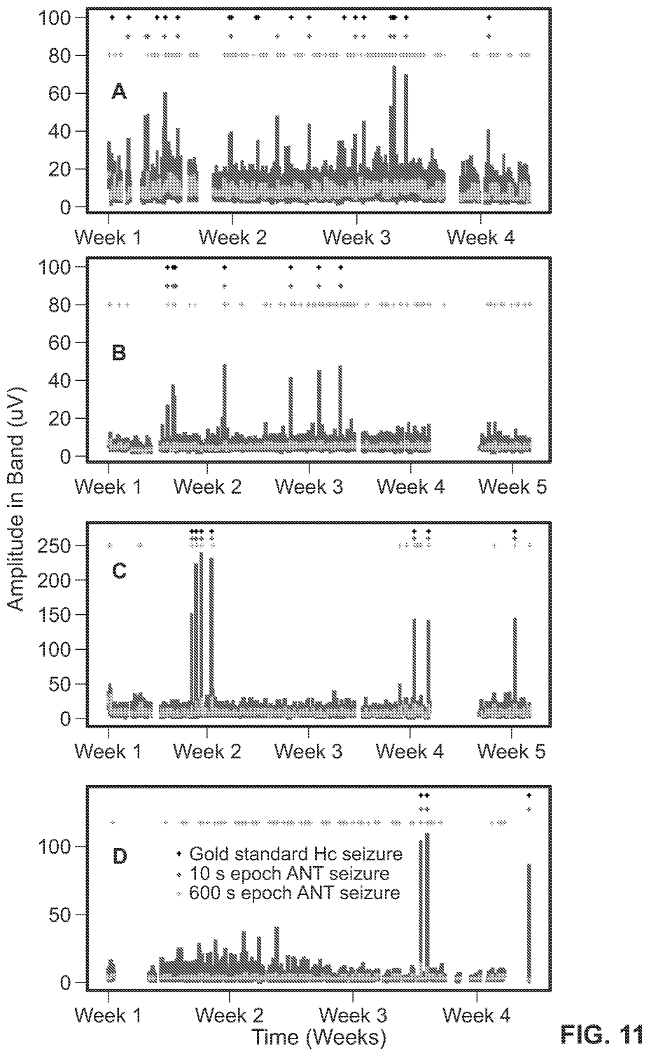

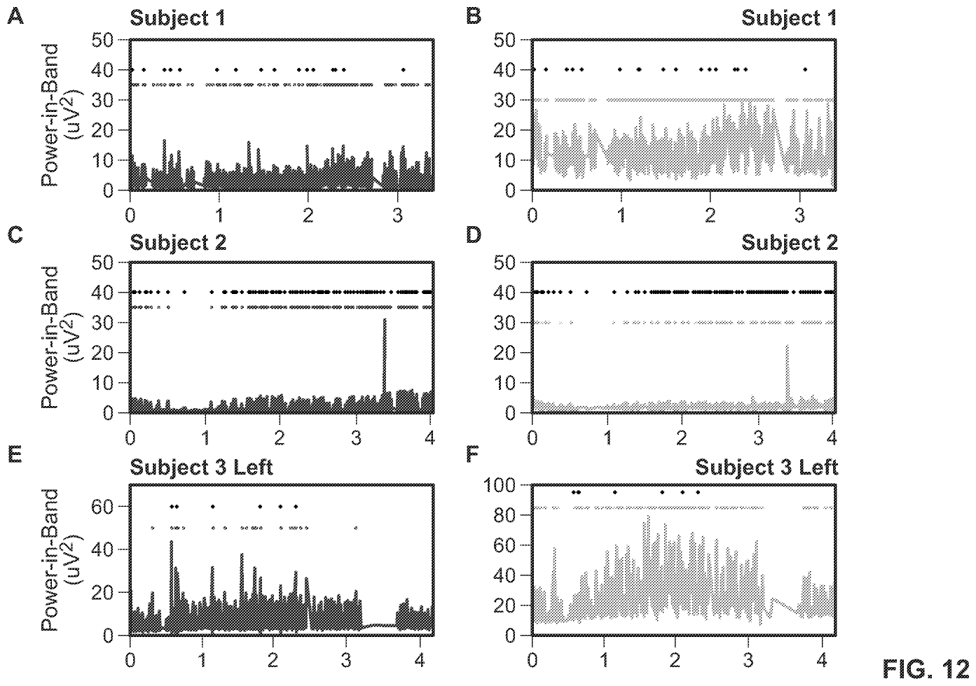

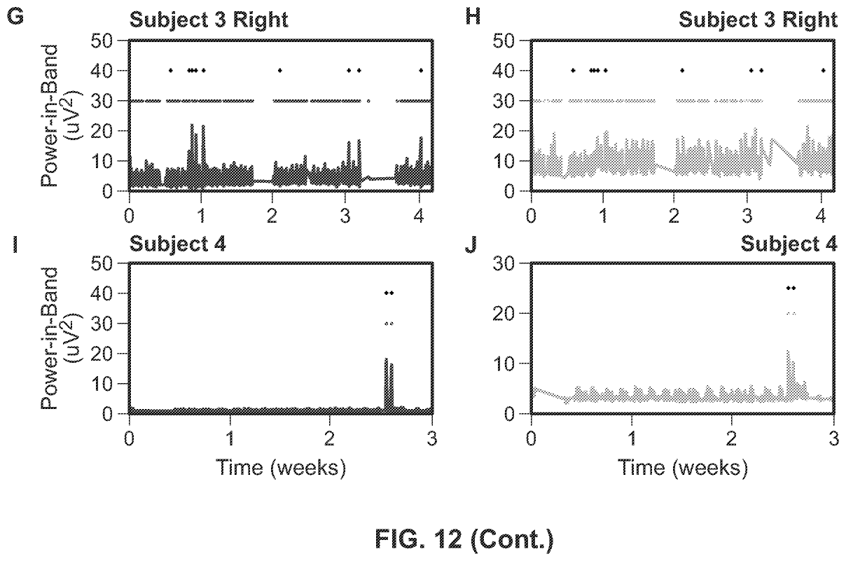

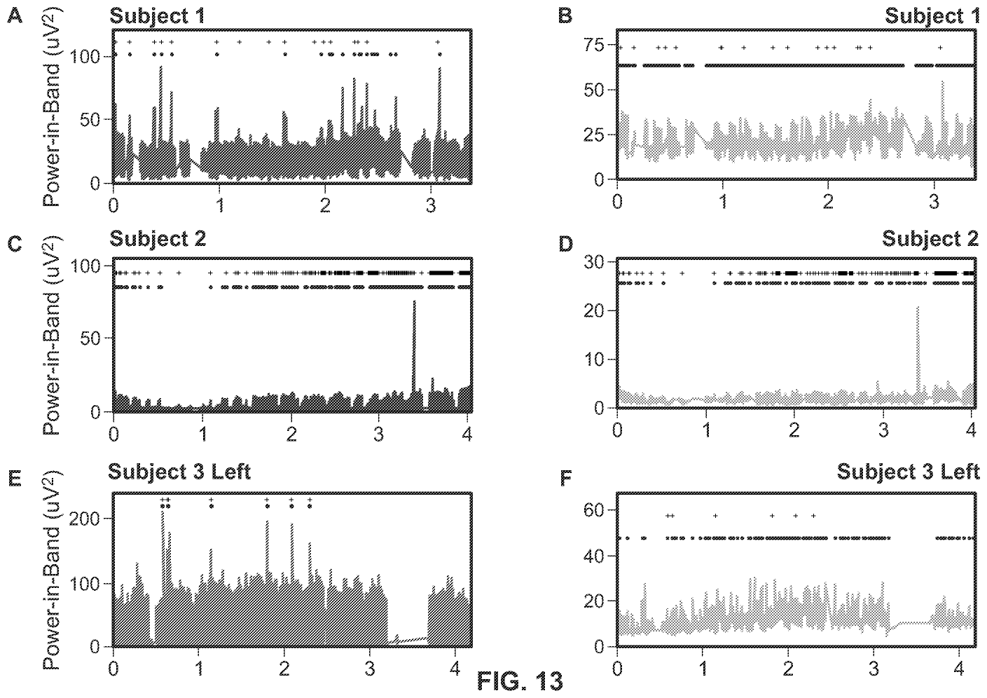

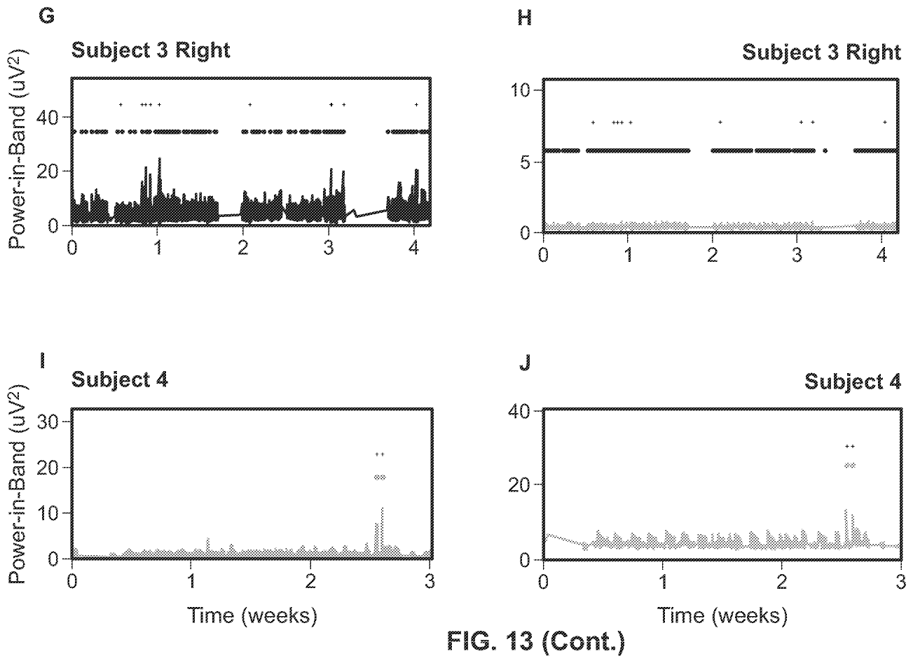

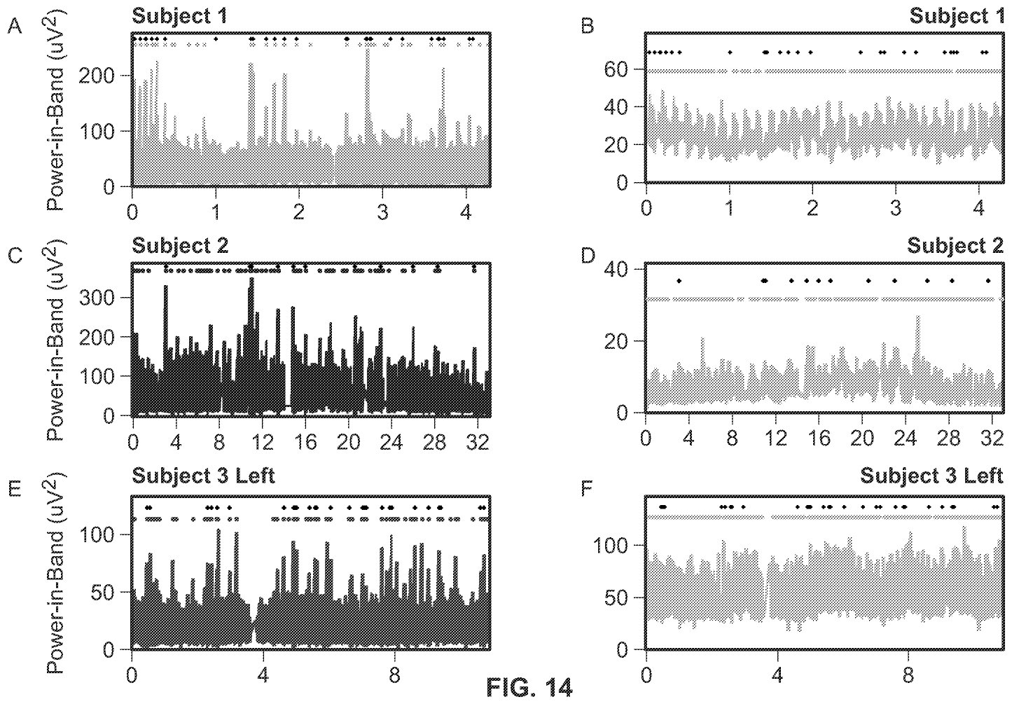

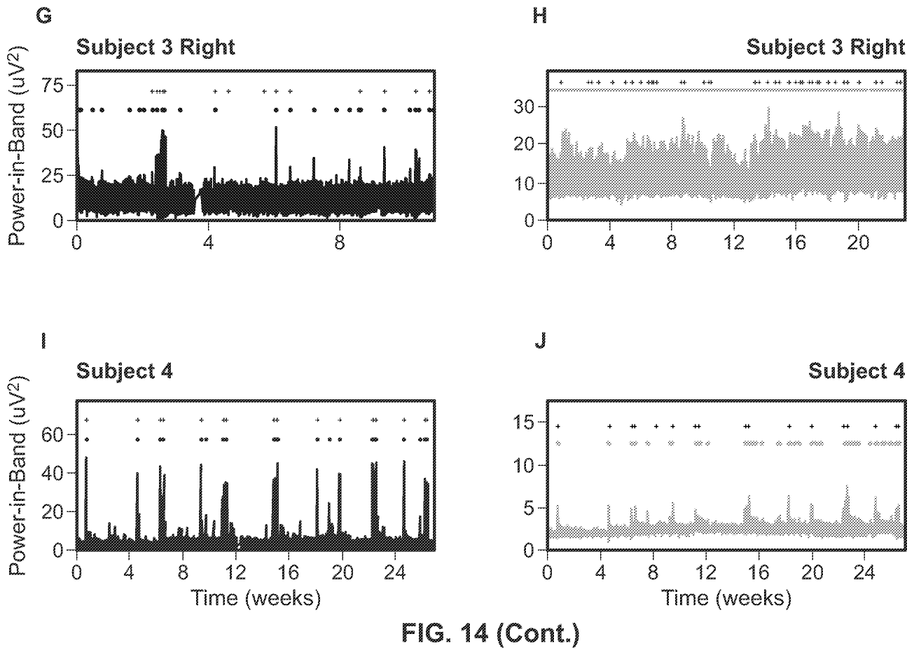

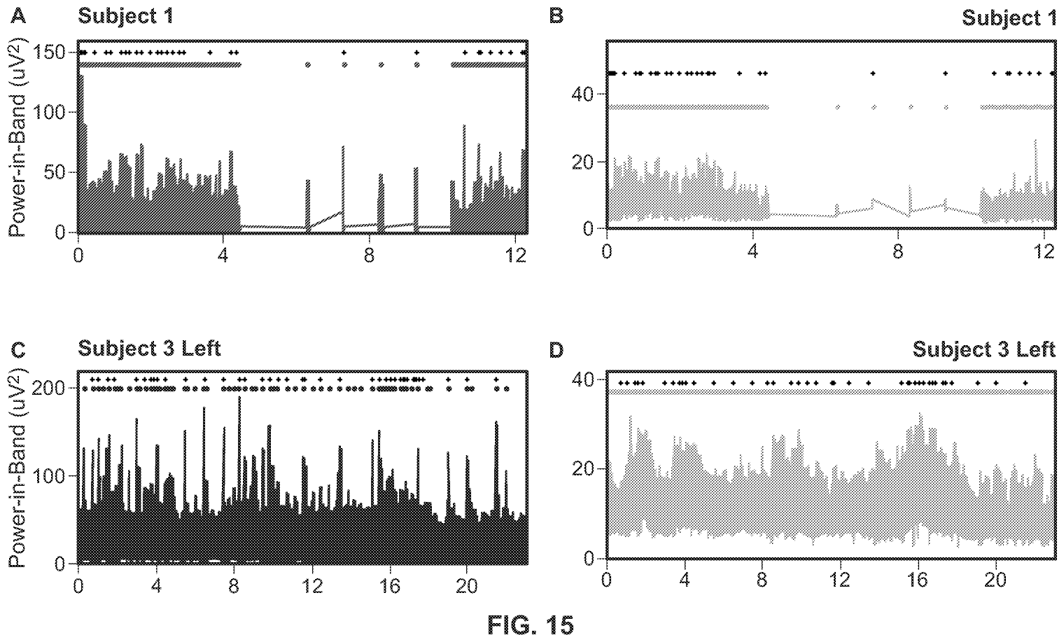

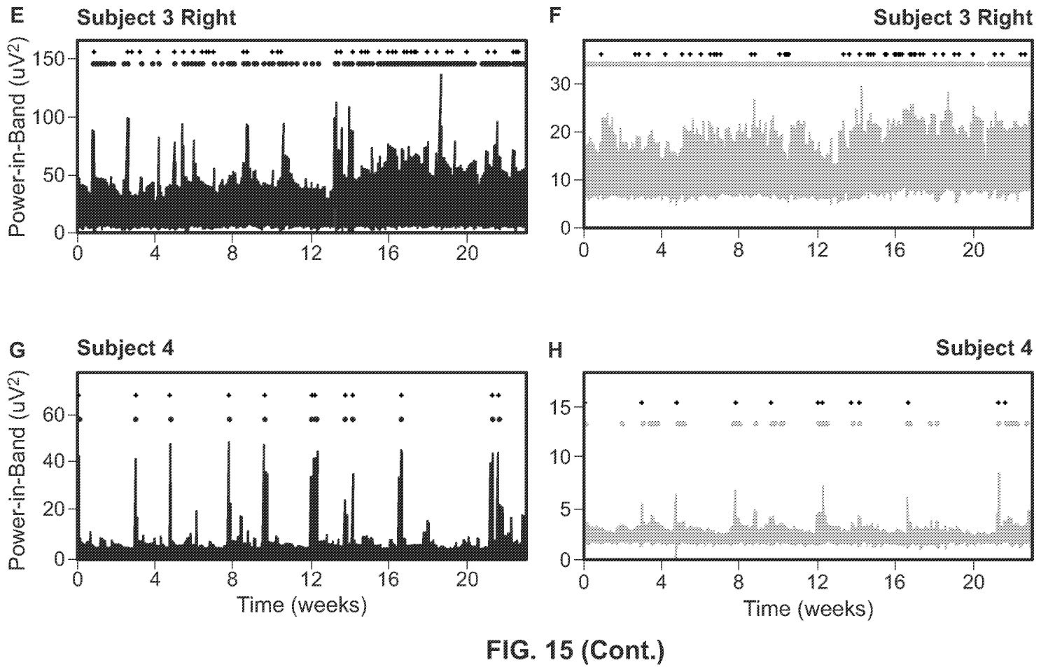

Notably, the PIB computations and thresholding techniques can be performed with relatively little processing expense or power consumption, which is a critical consideration for implanted devices 102 if chronic monitoring is to be performed. The performance of the classifier 120 will also vary widely as a function of the time and frequency parameters employed in different operating conditions. The values of the time and frequency parameters defined by the configuration profiles can thus be selected so as to yield effective classifier performance, e.g., through use of parameter values that have been empirically determined in studies to yield effective results. One measure of the classifier performance is area under the precision-recall curve (AU-PRC). Studies described herein (e.g., with respect to FIGS. 3A-3RRRR) have demonstrated an ability to achieve AU-PRC values of greater than 0.5 even in the presence of electrical brain stimulation. In this specification, effective classifier performance can be use-case specific. For example, high sensitivity can be prioritized for an automated seizure diary such as an electronic seizure log, but minimizing false positives could prioritized for responsive stimulation. Accordingly, effective classifier performance can be defined in a number of ways including AU-PRC of a least 0.5 or at least 0.6; sensitivity of at least 70%, at least 80%, at least 90%; or false positive rates less than 1 per day, less than 10 per day, or less than 100 per day when monitoring occurs for substantially the entire day (e.g., at least 18 hours, 20 hours, 23 hours, or all 24 hours of the day).

Seizure logging subsystem 124 maintains a log on the implantable device 102 of detected seizures. In some implementations, the log comprises a counter, and the logging subsystem 124 increments the counter each time a seizure is detected. Thus, the log may maintain a count of the number of seizures detected over a number of hours, days, weeks, or months, such as since a patient's last clinical appointment. In some implementations, the log can store further data about each recorded seizure event such as a timestamp indicating the date/time at which the seizure was detected to have occurred. The log can be transmitted to an external system on request or at scheduled times through a communication interface 128, such as a BLUETOOTH, near-field communications (NFC), or USB interface. In some implementations, each segment classified as seizure positive is logged as a detected seizure event. In some implementations, additional logic may be applied to correlate multiple iEEG segments (e.g., successive iEEG segments in a short time span) to a single seizure that would be recorded once in the log.

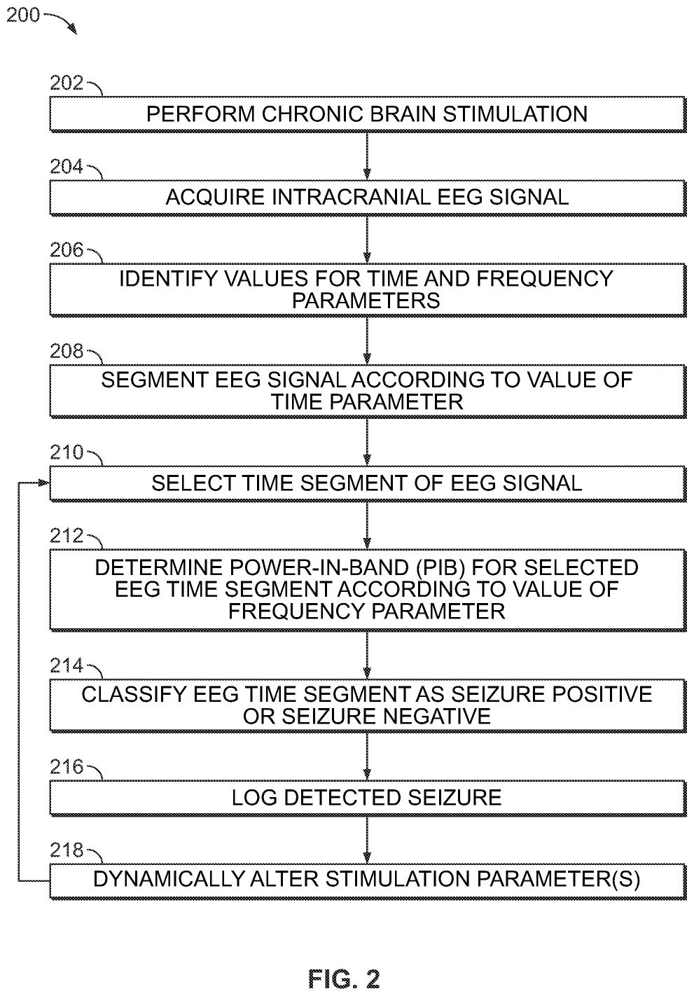

FIG. 2 is a flowchart of an example process 200 for detecting seizures using power-in-band classification techniques in the presence of electrical brain stimulation. A stimulating unit drives a set of stimulating electrodes to stimulate a region of a brain according to a prescribed therapy (202). A detection unit acquires an iEEG signal from one or more sensing electrodes in the brain (204). The iEEG signal can be acquired regardless of whether stimulation is off or ongoing. The process 200 identifies values for time and frequency parameters optimized for a given circumstance (e.g., whether no stimulation is applied, low-frequency stimulation is applied, or high-frequency stimulation is applied) (206). The EEG signal is segmented into fixed-time windows (208), and a particular segment is selected for processing (210). The power in a frequency band defined by the value of the frequency parameter is determined (212), and the segment is then classified as either seizure positive or seizure negative based on whether the in-band power in the segment meets or exceeds a threshold power level (214). If a seizure is detected based on the classification, it is automatically logged on the device (216). Further, in some embodiments, stimulation can be initiated or the manner of stimulation can be altered (218) in response to detecting the onset or end of a seizure (218).

The seizure detection techniques disclosed in this specification were evaluated in a study approved by the Food and Drug Administration (IDE G180224) and Mayo Clinic Institutional Review Board. The study was approved for up to ten subjects. Nine were consented, and four subjects (referred to as Subjects 1-4 throughout) were implanted with the investigational MEDTRONIC SUMMIT RC+S™ with leads targeting bilateral ANT and Hc (3387 and 3391 leads, with electrode span of 10.5 and 24 mm, respectively). The IPG provided continuous cLFP time series data sampled at 250 Hz from four bipolar pairs selected from four leads. Time series data were remotely streamed to a cloud-based server supporting data visualization, annotation, and computation. All analyses used RC+S™ ANT data acquired during the baseline stimulation-off period or RC+S™ Hc data acquired during either the baseline stimulation-off period, 2 Hz stimulation condition, or 100 Hz stimulation condition.

FIGS. 3A-3RRRR are plots that describe the performance of a classifier for a seizure detector in the present study as measured by area under the precision-recall curve (AU-PRC) according to various time and frequency parameter values.

The combination of parameter values corresponding to each of the plots are identified in Table 1 below. In this study, a power-in-band (PIB) thresholding technique was used to distinguish between seizure and non-seizure states. The algorithm was programmed to be sufficiently compact in terms of memory consumption and processing expense in order to permit use on a relatively low-powered implantable device. An investigational MEDTRONIC® SUMMIT RC+S™ was employed for this particular study, although it is contemplated that other systems such as the MEDTRONIC® PERCEPT PC+S™ could also be adapted to implement the seizure detection techniques described herein. In this study, four patients were implanted with leads having sensing electrodes in bilateral hippocampi (Hc) and stimulating electrodes in anterior thalamic nuclei (ANT). Long-term, ambulatory intracranial EEG (iEEG) data (scored by board-certified epileptologists) was collected and implemented an in silico, Python mockup of the embedded detector. An Hc seizure classifier was optimized with a grid search of PIB parameters: center frequency (CF; 3-98 Hz), bandwidth (BW; 5-40 Hz), and time window (2-600s). Area under the precision-recall curve (AU-PRC) was used to evaluate performance. Analysis was performed for three thalamic stimulation conditions: none, 2 Hz, and 145 Hz. One month of iEEG was recorded from each stimulation condition, analyzing each hemisphere independently.

TABLE 1

| | Stim. | | Time |
|---|---|---|---|
| FIG. | Frequency (Hz) | Bandwidth (Hz) | Window (s) |
| 3A | 0 | 5 | 2 |
| 3B | 0 | 5 | 5 |
| 3C | 0 | 5 | 10 |
| 3D | 0 | 5 | 20 |
| 3E | 0 | 5 | 30 |
| 3F | 0 | 5 | 60 |
| 3G | 0 | 5 | 120 |
| 3H | 0 | 5 | 600 |
| 3I | 0 | 10 | 2 |
| 3J | 0 | 10 | 5 |
| 3K | 0 | 10 | 10 |
| 3L | 0 | 10 | 20 |
| 3M | 0 | 10 | 30 |
| 3N | 0 | 10 | 60 |
| 3O | 0 | 10 | 120 |
| 3P | 0 | 10 | 600 |
| 3Q | 0 | 20 | 2 |
| 3R | 0 | 20 | 5 |
| 3S | 0 | 20 | 10 |
| 3T | 0 | 20 | 20 |
| 3U | 0 | 20 | 30 |
| 3V | 0 | 20 | 60 |
| 3W | 0 | 20 | 120 |
| 3X | 0 | 20 | 600 |
| 3Y | 0 | 40 | 2 |
| 3Z | 0 | 40 | 5 |
| 3AA | 0 | 40 | 10 |
| 3BB | 0 | 40 | 20 |
| 3CC | 0 | 40 | 30 |
| 3DD | 0 | 40 | 60 |
| 3EE | 0 | 40 | 120 |
| 3FF | 0 | 40 | 600 |
| 3GG | 2 | 5 | 2 |
| 3HH | 2 | 5 | 5 |
| 3II | 2 | 5 | 10 |
| 3JJ | 2 | 5 | 20 |
| 3KK | 2 | 5 | 30 |
| 3LL | 2 | 5 | 60 |
| 3MM | 2 | 5 | 120 |
| 3NN | 2 | 5 | 600 |
| 3OO | 2 | 10 | 2 |
| 3PP | 2 | 10 | 5 |
| 3QQ | 2 | 10 | 10 |
| 3RR | 2 | 10 | 20 |
| 3SS | 2 | 10 | 30 |
| 3TT | 2 | 10 | 60 |
| 3UU | 2 | 10 | 120 |
| 3VV | 2 | 10 | 600 |
| 3WW | 2 | 20 | 2 |
| 3XX | 2 | 20 | 5 |
| 3YY | 2 | 20 | 10 |
| 3ZZ | 2 | 20 | 20 |
| 3AAA | 2 | 20 | 30 |
| 3BBB | 2 | 20 | 60 |
| 3CCC | 2 | 20 | 120 |
| 3DDD | 2 | 20 | 600 |
| 3EEE | 2 | 40 | 2 |
| 3FFF | 2 | 40 | 5 |
| 3GGG | 2 | 40 | 10 |
| 3HHH | 2 | 40 | 20 |
| 3III | 2 | 40 | 30 |
| 3JJJ | 2 | 40 | 60 |
| 3KKK | 2 | 40 | 120 |
| 3LLL | 2 | 40 | 600 |
| 3MMM | 145 | 5 | 2 |
| 3NNN | 145 | 5 | 5 |
| 3OOO | 145 | 5 | 10 |
| 3PPP | 145 | 5 | 20 |
| 3QQQ | 145 | 5 | 30 |
| 3RRR | 145 | 5 | 60 |
| 3SSS | 145 | 5 | 120 |
| 3TTT | 145 | 5 | 600 |
| 3UUU | 145 | 10 | 2 |
| 3VVV | 145 | 10 | 5 |
| 3WWW | 145 | 10 | 10 |
| 3XXX | 145 | 10 | 20 |
| 3YYY | 145 | 10 | 30 |
| 3ZZZ | 145 | 10 | 60 |
| 3AAAA | 145 | 10 | 120 |
| 3BBBB | 145 | 10 | 600 |
| 3CCCC | 145 | 20 | 2 |
| 3DDDD | 145 | 20 | 5 |
| 3EEEE | 145 | 20 | 10 |
| 3FFFF | 145 | 20 | 20 |
| 3GGGG | 145 | 20 | 30 |
| 3HHHH | 145 | 20 | 60 |
| 3IIII | 145 | 20 | 120 |
| 3JJJJ | 145 | 20 | 600 |
| 3KKKK | 145 | 40 | 2 |
| 3LLLL | 145 | 40 | 5 |
| 3MMMM | 145 | 40 | 10 |
| 3NNNN | 145 | 40 | 20 |
| 3OOOO | 145 | 40 | 30 |
| 3PPPP | 145 | 40 | 60 |
| 3QQQQ | 145 | 40 | 120 |
| 3RRRR | 145 | 40 | 600 |

In this study, it was demonstrated that that when stimulation is off (0 Hz stimulation frequency), the classifier performed best with a 35 Hz center frequency, 40 Hz bandwidth, and 30 second window for the time segments with hippocampal sensing (AU-PRC=0.692, 91.2% sensitivity, and 63.9 false positives per day). The study further demonstrated that, in the presence of 2 Hz thalamic stimulation, the classifier performed best with a 5 Hz center frequency, 5 Hz bandwidth, and 20 second time segment window with hippocampal sensing (AU-PRC-0.571, 89% sensitivity, and 0.715 false positives per day). In the presence of 145 Hz thalamic stimulation, the classifier performed best with a 17 Hz center frequency, 5 Hz bandwidth, and 20 second time segment window with hippocampal sensing (AU-PRC=0.626, 88.6% sensitivity, and 47 false positives per day).

In more detail, the study demonstrated that the following combinations of time and frequency parameters yielded an AU-PRC value of at least 0.5:

TABLE 2

| Stim. | | Time | Range of Center |
|---|---|---|---|
| Frequency (Hz) | Bandwidth (Hz) | Window (s) | Frequencies (Hz) |
| 0 | 5 | 2 | 15-19 |
| 0 | 5 | 5 | 5, 13-21, 27-29 |
| 0 | 5 | 10 | 3-7, 13-31 |
| 0 | 5 | 20 | 5-9, 13-33, 37-39 |
| 0 | 5 | 30 | 5-39, 45-47 |
| 0 | 5 | 60 | 13-39 |
| 0 | 5 | 120 | 15-25, 31 |
| 0 | 10 | 2 | 18-20 |
| 0 | 10 | 5 | 8-28 |
| 0 | 10 | 10 | 8-32 |
| 0 | 10 | 20 | 8-36, 42-44 |
| 0 | 10 | 30 | 8-44, 48-50 |
| 0 | 10 | 60 | 10-44 |
| 0 | 10 | 120 | 14-24, 28 |
| 0 | 20 | 5 | 11-31 |
| 0 | 20 | 10 | 11-33 |
| 0 | 20 | 20 | 11-45, 53 |
| 0 | 20 | 30 | 11-49 |
| 0 | 20 | 60 | 11-45 |
| 0 | 20 | 120 | 19-31 |
| 0 | 40 | 5 | 21-39 |
| 0 | 40 | 10 | 21-45 |
| 0 | 40 | 20 | 21-49 |
| 0 | 40 | 30 | 21-51 |
| 0 | 40 | 60 | 21-49 |
| 0 | 40 | 120 | 25-37 |
| 2 | 5 | 5 | 5 |
| 2 | 5 | 10 | 5 |
| 2 | 5 | 20 | 5 |
| 2 | 5 | 30 | 5, 17 |
| 2 | 10 | 20 | 8 |
| 2 | 10 | 30 | 6-8, 20 |
| 2 | 20 | 20 | 11 |
| 2 | 20 | 30 | 11-13 |
| 2 | 40 | 30 | 21-23 |
| 145 | 5 | 5 | 15-17 |
| 145 | 5 | 10 | 15-19 |
| 145 | 5 | 20 | 15-19 |
| 145 | 5 | 30 | 15-21 |
| 145 | 10 | 10 | 18-20 |
| 145 | 10 | 20 | 16-22 |
| 145 | 10 | 30 | 18-22 |
| 145 | 20 | 10 | 23-25 |
| 145 | 20 | 20 | 21-25 |
| 145 | 20 | 30 | 25 |

Table 3 below describes data from the study indicating generalized, optimized detector performance for each patient with leads in Hc during either 2 Hz or 145 Hz ANT stimulation. Patient 2 did not receive 145 Hz stimulation due to poor tolerance.

TABLE 3

| | Patient | | | | | | | | | | | | | |
|---|---|---|---|---|---|---|---|---|---|---|---|---|---|---|
| | | | | Subject | Subject | | | | | | | | | |
| | | | | 2 Left | 2 Right | | | | | | | | | |
| | Subject 1 Left Hc | | | Hc | Hc | Subject 3 Left Hc | | | Subject 3 Right Hc | | | Subject 4 Left Hc | | |
| | Stimulation Frequency | | | | | | | | | | | | | |
| | 0 Hz | 2 Hz | 145 Hz | 0 Hz | 2 Hz | 0 Hz | 2 Hz | 145 Hz | 0 Hz | 2 Hz | 145 Hz | 0 Hz | 2 Hz | 145 Hz |
| AU-PRC | 0.248 | 0.564 | 0.512 | 0.712 | 0.653 | 0.721 | 0.464 | 0.666 | 0.778 | 0.331 | 0.477 | 1 | 0.846 | 0.85 |
| Sensitivity | 80.0% | 89.3% | 80.3% | 88.3% | 92.3% | 100.0% | 86.7% | 93.6% | 88.9% | 81.0% | 80.5% | 100.0% | 100.0% | 100.0% |
| FP/Day | 61.6 | 207.8 | 86.3 | 9.96 | 0.81 | 0.751 | 0.97 | 0.951 | 252.7 | 0.799 | 101 | 0 | 0.81 | 0.0186 |

Table 4 below describes data from the study indicating the individualized optimal set of parameters for each patient without stimulation on.

TABLE 4

| | Patient | | | | | | | | | |
|---|---|---|---|---|---|---|---|---|---|---|
| | Subject | | Subject | | Subject | | Subject | | Subject | |
| | 1 | | 2 Left | | 3 Left | | 3 Right | | 4 | |
| Bandwidth | 5 | Hz | 10 | Hz | 5 | Hz | 5 | Hz | 5 | Hz |
| Time Window | 10 | s | 30 | s | 2 | s | 20 | s | 5 | s |
| Center Frequency | 5 | Hz | 14 | Hz | 19 | Hz | 27 | Hz | 47 | Hz |

Table 5 below describes data from the study indicating performance of the hippocampal detector with individualized, optimized parameter sets and no ANT stimulation. When sensitivity was allowed to drop as low as 70%, the number of false positives was greatly reduced.

TABLE 5

| | Patient | | | | |
|---|---|---|---|---|---|
| | Subject | Subject | Subject | Subject | Subject |
| | 1 | 2 Left | 3 Left | 3 Right | 4 |
| AUPRC | 0.575 | 0.82 | 0.889 | 0.78 | 1 |
| Sensitivity >80% | 80% | 85.9% | 100% | 88.9% | 100% |
| FP/day | 0.885 | 1.91 | 0 | 43.9 | 0 |
| Sensitivity >70% | 80% | 74.5% | 100% | 77.7% | 100% |
| FP/day | 0.885 | 1.7 | 0 | 0.034 | 0 |

Table 6 below describes data from the study indicating performance of the hippocampal detector with commercial parameter sets and no ANT stimulation. When sensitivity was allowed to drop as low as 70%, the number of false positives was greatly reduced.

TABLE 6

| | Patient | | | | |
|---|---|---|---|---|---|
| | Subject | Subject | Subject | Subject | Subject |
| | 1 | 2 Left | 3 Left | 3 Right | 4 |
| AUPRC | 0.018 | 0.621 | 0.00964 | 0.0515 | 1 |
| Sensitivity >80% | 84% | 85.5% | 88.9% | 88.9% | 100% |
| FP/day | 108.9 | 8.83 | 16.38 | 82.76 | 0 |
| Sensitivity >70% | 73.7% | 71.4% | 88.9% | 77.8% | 100% |
| FP/day | 100 | 4.36 | 16.38 | 16.21 | 0 |

Table 7 below describes data from the study indicating the individualized optimal set of parameters for each patient during 2 Hz ANT stimulation.

TABLE 7

| | Patient | | | | | | | | | |
|---|---|---|---|---|---|---|---|---|---|---|
| | Subject | | Subject | | Subject | | Subject | | Subject | |
| | 1 | | 2 Right | | 3 Left | | 3 Right | | 4 | |
| Bandwidth | 5 | Hz | 5 | Hz | 5 | Hz | 5 | Hz | 40 | Hz |
| Time Window | 5 | s | 20 | s | 30 | s | 60 | s | 20 | s |
| Center Frequency | 3 | Hz | 5 | Hz | 19 | Hz | 17 | Hz | 27 | Hz |

Table 8 below describes data from the study indicating performance of the hippocampal detector with individualized, optimized parameter sets and 2 Hz ANT stimulation. When sensitivity was allowed to drop as low as 70%, the number of false positives was unchanged.

TABLE 8

| | Patient | | | | |
|---|---|---|---|---|---|
| | Subject | Subject | Subject | Subject | Subject |
| | 1 | 2 Right | 3 Left | 3 Right | 4 |
| AUPRC | 0.755 | 0.636 | 0.652 | 0.587 | 0.993 |
| Sensitivity >80% | 82% | 92.3% | 100% | 81.0% | 100% |
| FP/day | 0.9 | 0.81 | 0.652 | 0.996 | 0.0529 |
| Sensitivity >70% | 82% | 92.3% | 100% | 81.0% | 100% |
| FP/day | 0.9 | 0.81 | 0.652 | 0.996 | 0.0529 |

Table 9 below describes data from the study indicating performance of the hippocampal detector with commercial parameter sets and 2 Hz ANT stimulation. When sensitivity was allowed to drop as low as 70%, the number of false positives was slightly reduced.

TABLE 9

| | Patient | | | | |
|---|---|---|---|---|---|
| | Subject | Subject | Subject | Subject | Subject |
| | 1 | 2 Right | 3 Left | 3 Right | 4 |
| AUPRC | 0.00608 | 0.00196 | 0.00496 | 0.0221 | 0.772 |
| Sensitivity >80% | 81.5% | 84.6% | 80% | 81.0% | 82.8% |
| FP/day | 103.5 | 25.3 | 54.3 | 31.3 | 0.82 |
| Sensitivity >70% | 70.4% | 84.6% | 70% | 76% | 82.8% |
| FP/day | 92 | 25.3 | 45.2 | 20.5 | 0.82 |

Table 10 below describes data from the study indicating the individualized optimal set of parameters for each patient during 145 Hz ANT stimulation.

TABLE 10

Patient

| | Subject | | Subject | | Subject | | Subject | |
|---|---|---|---|---|---|---|---|---|
| | 1 | | 3 Left | | 3 Right | | 4 | |
| Bandwidth | 5 | Hz | 5 | Hz | 5 | Hz | 10 | Hz |
| Time Window | 10 | s | 20 | s | 20 | s | 20 | s |
| Center Frequency | 17 | Hz | 17 | Hz | 5 | Hz | 22 | Hz |

Table 11 below describes data from the study indicating performance of the hippocampal detector with individualized, optimized parameter sets and 145 Hz ANT stimulation. When sensitivity was allowed to drop as low as 70%, the number of false positives was greatly reduced.

TABLE 11

Patient

| | Subject | Subject | Subject | Subject |
|---|---|---|---|---|
| | 1 | 3 Left | 3 Right | 4 |
| AUPRC | 0.512 | 0.349 | 0.56 | 0.904 |
| Sensitivity >80% | 80.3% | 93.6% | 80.5% | 100% |
| FP/day | 75.4 | 0.95 | 7.32 | 0.0186 |
| Sensitivity >70% | 71.9% | 93.6% | 70.1% | 100% |
| FP/day | 39.5 | 0.95 | 2.25 | 0.0186 |

Table 12 below describes data from the study indicating performance of the hippocampal detector with commercial parameter sets and 145 Hz ANT stimulation. When sensitivity was allowed to drop as low as 70%, the number of false positives was slightly reduced.

TABLE 12

Patient

| | Subject | Subject | Subject | Subject |
|---|---|---|---|---|
| | 1 | 3 Left | 3 Right | 4 |
| AUPRC | 0.00978 | 0.004 | 0.0136 | 0.522 |
| Sensitivity >80% | 81.3% | 80.4% | 81.8% | 87.5% |
| FP/day | 51.5 | 53.8 | 54.1 | 0.795 |
| Sensitivity >70% | 71.9% | 71.7% | 72.7% | 87.5% |
| FP/day | 39.5 | 40 | 44.5 | 0.795 |

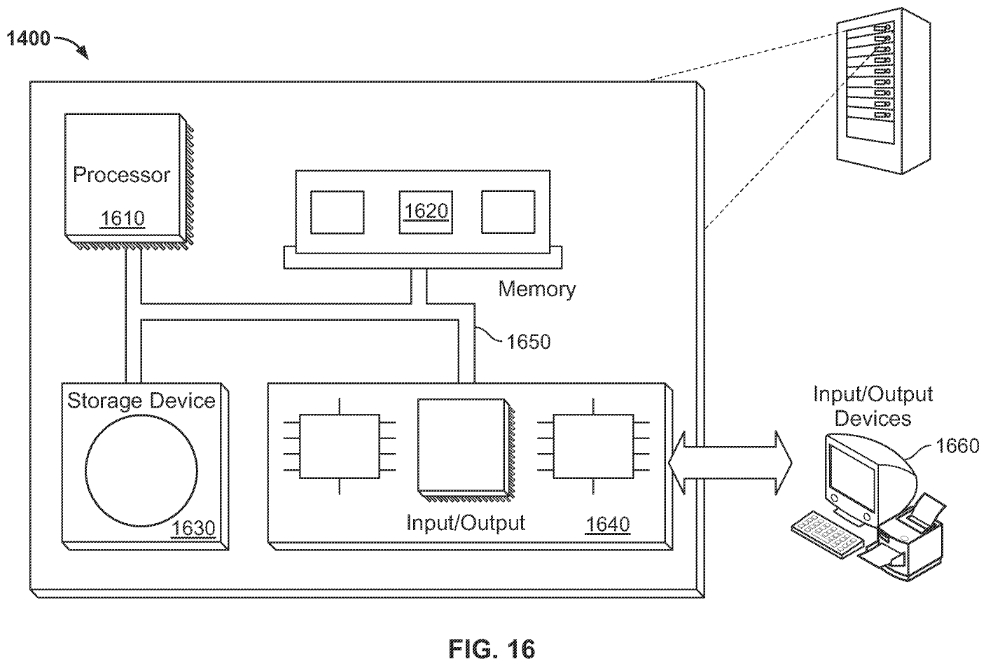

FIG. 16 shows an example computer system 1600 that includes a processor 1600, a memory 1620, a storage device 1630 and an input/output device 1640. Each of the components 1610, 1620, 1630 and 1640 can be interconnected, for example, by a system bus 1650. The processor 1610 is capable of processing instructions for execution within the system 1600. In some implementations, the processor 1610 is a single-threaded processor, a multi-threaded processor, or another type of processor. The processor 1610 is capable of processing instructions stored in the memory 1620 or on the storage device 1630. The memory 1620 and the storage device 1630 can store information within the system 1600.

The input/output device 1640 provides input/output operations for the system 1600. In some implementations, the input/output device 1640 can include one or more of a network interface device, e.g., an Ethernet card, a serial communication device, e.g., an RS-232 port, and/or a wireless interface device, e.g., an 802.11 card, a 3G wireless modem, a 4G wireless modem, a 16G wireless modem, etc. In some implementations, the input/output device can include driver devices configured to receive input data and send output data to other input/output devices, e.g., keyboard, printer and display devices 1660. In some implementations, mobile computing devices, mobile communication devices, and other devices can be used.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of detecting seizures in a mammal, the method comprising:
stimulating, with a first set of electrodes in a brain of the mammal, a region of the brain;
while stimulating the region of the brain, acquiring an electrical signal sensed by a second set of electrodes in the brain;
identifying values for a time parameter and a frequency parameter to be used in signal power calculations;
segmenting the electrical signal into a plurality of time-segmented portions according to a value of the time parameter;
for each time-segmented portion of the electrical signal:
determining a power of the time-segmented portion of the electrical signal within a frequency band defined by a value of the frequency parameter; and
using a classifier to classify the time-segmented portion of the electrical signal as seizure positive or seizure negative based on the determined power within the frequency band;
wherein the values for the time parameter and the frequency parameter result in the classifier achieving an effective level of performance defined by an area under a precision-recall curve (AU-PRC) for the classifier of at least 0.5.

2. The method of claim 1, wherein the mammal is a human.

3. The method of claim 1, wherein the region of the brain stimulated with the first set of electrodes is a region of the thalamus.

4. The method of claim 3, wherein the region of the brain stimulated with the first set of electrodes comprises an anterior nuclei of the thalamus.

5. The method of claim 3, wherein the second set of electrodes produces the electrical signal responsive to brain activity in the hippocampus.

6. The method of claim 1, wherein identifying values for the time parameter and the frequency parameter comprises selecting a set of values for the time parameter and the frequency parameter that are associated with a frequency with which the first set of electrodes stimulates the region of the brain, wherein different sets of values for the time parameter and the frequency parameter are associated with different stimulation frequencies.

7. The method of claim 6, wherein a first set of values for the time parameter and the frequency parameter are associated with a low-frequency stimulation and a second set of values for the time parameter and the frequency parameter are associated with a high-frequency stimulation.

8. (canceled)

9. The method of claim 6, comprising:
adjusting the frequency with which the first set of electrodes stimulates the region of the brain from a first frequency to a second frequency; and
in response to the adjusting, applying a second set of values for the time parameter and the frequency parameter to segment the electrical signal and determine powers of time-segmented portions of the electrical signal in place of a first set of values for the time parameter and the frequency parameter, wherein the second set of values is associated with the second frequency and the first set of values is associated with the first frequency.

10. The method of claim 1, wherein stimulating the region of the brain comprises applying a chronic deep brain stimulation therapy to the region of the brain.

11. The method of claim 1, wherein the classifier is implemented on a device implanted in a body of the mammal.

12. The method of claim 11, wherein the device includes a memory and is configured to maintain a log of seizures detected over time, wherein the device logs a seizure event in response to the classifier classifying one or more time-segmented portions of the electrical signal as seizure positive.

13-14. (canceled)

15. The method of claim 1, wherein the value for the time parameter is a temporal segment length that defines a temporal length of the time-segmented portions of the electrical signal.

16. The method of claim 1, wherein the value for the frequency parameter comprises a center-frequency component and a bandwidth component.

17. The method of claim 1, wherein the value for the frequency parameter comprises an upper cutoff frequency component and a lower cutoff frequency component.

18. The method of claim 1, wherein using the classifier to classify the time-segmented portion of the electrical signal comprises comparing the determined power of the time-segmented portion of the electrical signal within the frequency band to a threshold power value.

19. The method of claim 1, comprising, in response to detecting a seizure event based on identification of one or more seizure positive time-segmented portions of the electrical signal, adjusting one or more parameters by which the first set of electrodes stimulate the region of the brain.

20. The method of claim 1, comprising, in response to detecting a seizure event based on identification of one or more seizure positive time-segmented portions of the electrical signal, initiating stimulation of a second region of the brain different from the region of the brain stimulated by the first set of electrodes.

21-22. (canceled)

23. A method of detecting seizures in a mammal, the method comprising:
stimulating, with a first set of electrodes in a brain of the mammal, a region of the brain;
while stimulating the region of the brain, acquiring an electrical signal sensed by a second set of electrodes in the brain;
identifying values for a time parameter and a frequency parameter to be used in signal power calculations;
segmenting the electrical signal into a plurality of time-segmented portions according to a value of the time parameter;
for each time-segmented portion of the electrical signal:
determining a power of the time-segmented portion of the electrical signal within a frequency band defined by a value of the frequency parameter;
using a classifier to classify the time-segmented portion of the electrical signal as seizure positive or seizure negative based on the determined power within the frequency band; and
logging, in memory of a device implanted in the mammal, an indication of each seizure event detected in the mammal over a period of time, each seizure event corresponding to one or more time-segmented portions of the electrical signal that were classified as seizure positive.

24. A system comprising (a) circuitry configured to perform a method of detecting seizures in a mammal, wherein the method comprises (a1):
stimulating, with a first set of electrodes in a brain of the mammal, a region of the brain;
while stimulating the region of the brain, acquiring an electrical signal sensed by a second set of electrodes in the brain;
identifying values for a time parameter and a frequency parameter to be used in signal power calculations;
segmenting the electrical signal into a plurality of time-segmented portions according to a value of the time parameter;
for each time-segmented portion of the electrical signal:
determining a power of the time-segmented portion of the electrical signal within a frequency band defined by a value of the frequency parameter; and
using a classifier to classify the time-segmented portion of the electrical signal as seizure positive or seizure negative based on the determined power within the frequency band;
wherein the values for the time parameter and the frequency parameter result in the classifier achieving an effective level of performance defined by an area under a precision-recall curve (AU-PRC) for the classifier of at least 0.5,
or wherein the method comprises (a2):
stimulating, with a first set of electrodes in a brain of the mammal, a region of the brain;
while stimulating the region of the brain, acquiring an electrical signal sensed by a second set of electrodes in the brain;
identifying values for a time parameter and a frequency parameter to be used in signal power calculations;
segmenting the electrical signal into a plurality of time-segmented portions according to a value of the time parameter;
for each time-segmented portion of the electrical signal:
determining a power of the time-segmented portion of the electrical signal within a frequency band defined by a value of the frequency parameter;
using a classifier to classify the time-segmented portion of the electrical signal as seizure positive or seizure negative based on the determined power within the frequency band; and
logging, in memory of a device implanted in the mammal, an indication of each seizure event detected in the mammal over a period of time, each seizure event corresponding to one or more time-segmented portions of the electrical signal that were classified as seizure positive; or
(b) one or more stimulating electrodes disposed in a brain of a mammal;
one or more sensing electrodes disposed in a brain of the mammal;
a stimulation unit configured to generate and deliver stimulation signals to the stimulating electrodes to cause the stimulating electrodes to stimulate a region of a brain of the mammal;
a seizure detection unit, comprising:
memory storing one or more sets of values for a time parameter and a frequency parameter;
signal acquisition circuitry configured to acquire an electroencephalogram (EEG) signal sensed by the one or more sensing electrodes;
a signal segmentation engine configured to segment the EEG signal into a plurality of time-segmented portions according to a value of a time parameter;
a power analyzer configured, for each time-segmented portion of the EEG signal, to determine a power of the time-segmented portion within a frequency band defined by a value of a frequency parameter; and
a classifier configured, for each time-segmented portion of the EEG signal, to classify the time-segmented portion as seizure positive or seizure negative based on the determined power of the time-segmented power within the frequency band,
wherein the values for the time parameter and the frequency parameter result in the classifier achieving an effective level of performance defined by an area under a precision-recall curve (AU-PRC) for the classifier of at least 0.5.

25-26. (canceled)
